SEQUENCE LISTING (20 sequences)

SEQ ID NO: 1 - DNA, 1650 bp, Artificial Sequence
  Synthetic lipase from C. cylindracea; ctg codon substituted for proper expression in plants
atggagctcgccctcgccctcagcctcatcgccagcgtcgccgccgccccgaccgccaccctcgccaacggcgacaccatcaccggcctcaacgccatcatcaacgaggccttcctcggcatcccgttcgccgagccgccggtcggcaacctccgcttcaaggacccggtcccgtacagcggcagcctcgacggccagaagttcaccagctacggcccgagctgcatgcagcagaacccggagggcacctacgaggagaacctcccgaaggccgccctcgacctcgtcatgcagagcaaggtcttcgaggccgtcagcccgagcagcgaggactgcctcaccatcaacgtcgtgcgccccccaggcactaaggccggcgccaatctgcctgtgatgctgtggatattcgggggcggcttcgaagtcggaggcacctcgacgttcccgcccgcccaaatgataacaaagtctatagcgatggggaagccaataatacacgtctcagtcaattacagggtcagtagctggggttttctcgctggagatgaaatcaaagcagagggctccgccaatgcggggttgaaagatcaaaggcttggtatgcaatgggtggctgataatattgcagcctttggaggcgatcctactaaagtcaccatatttggggaatcggcgggttctatgtcagttatgtgtcacatcctatggaacgacggagataatacgtacaaaggcaaaccgttatttcgcgctgggatcatgcaaagtggtgcaatggtacccagcgatgcggtcgatggaatctatggcaatgagatctttgatctgctcgcgtccaatgctgggtgcggttcggcatctgataagttggcctgccttcggggagtgtcaagtgatacactagaggatgcgactaataataccccaggcttcttagcttatagctccctgcgtctctcgtatttgcctcgaccggacggggtgaacattacggatgacatgtatgcacttgtgagagagggtaaatatgccaatattcccgtgattattggagaccagaacgacgagggcacattcttcgggacttcttcactaaacgtcaccacggacgcgcaagctagggagtactttaagcagagttttgttcatgcaagcgacgccgagattgacacattaatgactgcgtacccaggtgacattacccaaggatcccctttcgacacgggcatcctgaacgctctcacaccgcagtttaagcgcatctcggcagtattgggggaccttggtttcactctagcccggcgttatttcttaaaccactacaccggaggcacgaagtactctttcctgtcaaagcagctcagtggcttgcccgtgcttggtacattccacagcaacgacatcgtcttccaggactacctgctcggatccggctctcttatctacaataatgctttcattgctttcgctacggatcttgatccaaatacggctggtcttcttgttaagtggccagagtacacatcttcttctcagtctggtaataatcttatgatgattaatgctcttggtctttacacgggtaaggataatttcagaacagctggttacgatgctcttttctctaatccaccatctttcttcgtttag

SEQ ID NO: 2 - protein, 549 aa, Artificial Sequence
  Synthetic Lipase from C. cylindracea
MELALALSLIASVAAAPTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 3 - DNA, 1350 bp, Sus scrofa
  CDS (1)...(1350)
tccgaggtgtgcttcccgcgcctcggctgcttctccgacgacgccccgtgggccggcatcgtgcagcgcccgctcaagatcctcccgtggtccccgaaggacgtggacacccgcttcctcctctacaccaaccagaaccagaacaactaccaggagctcgtggccgacccgtccaccatcaccaactccaacttccgcatggaccgcaagacccgcttcatcatccacggcttcatcgacaagggcgaggaggactggctctccaacatctgcaagaacctcttcaaggtggagtccgtgaactgcatctgcgtggactggaagggcggctcccgcaccggctacacccaggcctcccagaacatccgcatcgtgggcgccgaggtggcctacttcgtggaggtgctcaagtcctccctcggctactccccgtccaacgtgcacgtgatcggccactccctcggctcccacgccgccggcgaggccggccgccgcaccaacggcaccatcgagcgcatcaccggcctcgacccggccgagccgtgcttccagggcaccccggagctcgtgcgcctcgacccgtccgacgccaagttcgtggacgtgatccacaccgacgccgccccgatcatcccgaacctcggcttcggcatgtcccagaccgtgggccacctcgacttcttcccgaacggcggcaagcagatgccgggctgccagaagaacatcctctcccagatcgtggacatcgacggcatctgggagggcacccgcgacttcgtggcctgcaaccacctccgctcctacaagtactacgccgactccatcctcaacccggacggcttcgccggcttcccgtgcgactcctacaacgtgttcaccgccaacaagtgcttcccgtgcccgtccgagggctgcccgcagatgggccactacgccgaccgcttcccgggcaagaccaacggcgtgtcccaggtgttctacctcaacaccggcgacgcctccaacttcgcccgctggcgctacaaggtgtccgtgaccctctccggcaagaaggtgaccggccacatcctcgtgtccctcttcggcaacgagggcaactcccgccagtacgagatctacaagggcaccctccagccggacaacacccactccgacgagttcgactccgacgtggaggtgggcgacctccagaaggtgaagttcatctggtacaacaacaacgtgatcaacccgaccctcccgcgcgtgggcgcctccaagatcaccgtggagcgcaacgacggcaaggtgtacgacttctgctcccaggagaccgtgcgcgaggaggtgctcctcaccctcaacccgtgc

SEQ ID NO: 4 - protein, 450 aa, Sus scrofa
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 5 - DNA, 3120 bp, Rhizopus arrhizus
  CDS (901)...(2079)
tatagtatagatactggtgagatagaacaaatggagcgcgtatacaaaataaatttagggtcatcttaaatttgagttcattatagggcctttttctgctgggaaaaggacacaaagttcgataacattcttggtcaatacaagataattgaatgcttgtgtttaatgagcttttatgctatttcatgatctattctagatcatgagataaacttatgtgctcaataaataaaattcttttcttaacaaagtctttaatttgatgaagtgatcaagtaatccttgtgccttataattgaaggatgatcaagtttgtgcttcaataaaataagttgcataatgcattggctttttatattttaataacatttctattaactcgaaatatctttcaaaataagcttcatatcaatttttgccttgtttcttccaactgcctacaacactaaattgaaataagtccggttttactttttcaatgggagaaaatggctgaattcttttgaaagttaagttatacattttcagctttactgtcgcacataaaattagtttattttatcccagcgagtgatataggaaaaatcagaattgtctcctttttttgtcttattttatgtaaaatccgctttgtgtgatgttttgtattacattcaaaaaaagaggaatcgctcgtaacaataattgatcacttggtactactattaaatatacctaatttcatgaggggttacaatgtgcgtggataaattgccattggtctctctattttttgaacaaaaaaaaacatataaatagagcaagtttatgttatgttcaagctctctatcttactaagctaattgatacagactcttcttttcttttcttcttaccccttccagttctttactatcaaacatggtttcattcatttccatttctcaaggtgttagtctttgtcttcttgtctcttccatgatgctcggttcatctgctgttcctgtttctggtaaatctggatcttccactaccgccgtctctgcatctgacaattctgccctccctcctctcatttccagccgttgtgctcctccttctaacaagggaagtaaaagcgatcttcaagctgaaccttactacatgcaaaagaatacagaatggtatgagtcccatggtggcaacctgacatccatcggaaagcgagatgacaatttggttggtggcatgactttggatttacctagcgatgctcctcctatcagcctctctggatctaccaacagcgcctctgatggtggtaaggttgttgctgctactactgctcaaattcaagagttcaccaagtatgctggtatcgctgccactgcctactgtcgttctgttgtccctggtaacaagtgggactgtgtccaatgtcaaaagtgggttcctgatggcaagatcatcactacctttacctccttgctttccgacacaaatggttacgtcttgagaagtgataaacaaaagaccatttatcttgttttccgtggtaccaactccttcagaagtgccatcactgatattgtcttcaacttttccgactacaagcctgtcaagggcgccaaggttcatgctggtttcctttcctcttatgagcaagttgtcaatgactatttccctgtcgtccaagaacaactgaccgccaaccctacttacaaggtcatcgtcaccggtcactcactcggtggtgcacaagctttgcttgccggtatggatctctaccaacgtgaaccaagactgtctcccaagaatttgagcatcttcactgttggtggtcctcgtgttggtaaccccacctttgcttactatgttgaatctaccggtattcctttccaacgtaccgttcacaagagagatatcgttcctcacgttcctcctcaatccttcggattccttcatcccggtgttgaatcttggattaagtctggtacctccaacgttcaaatctgtacttctgaaattgaaaccaaggattgcagtaactctatcgttcctttcacctctctccttgatcacttgagttactttgatatcaacgaaggaagctgtttgtaaaacacttgacgtgttactctaattttataataaaactaagtttttatacaataactttttgcatgtctacatataatttagaatgtaacctcaacttcaaacttgtatatcagtagtctcttatcatttcatctggtccatttttaaaactatgttcatagagtcatttacattagacatattctatgatatcctctgatctacagtcttcatttattcttttatgattcacgtaatgtcttgagtttagaaaaaatagtttaagagtttttttgtagttaaaaaattaatctctgcctttttttaggatttaaatattataatgttttacataacttgaaacccataccaaagtattttagtgttattttactaataaaataaaccttattgcttgtgaagccaattgatttttgtgcttatttcataaatttggttttatttagggaaagaaataacacaaggtgcaaagtagattgtttataaggaaaaggattgaaattgactagaacaaccatcaatattatttgcagagtagacatattaggctaatctgagttatctatcctctcgttatatttagcctaaaatgctgttattataagcattttgcagtatctgtaatttgctgaaatacttgcaagaaacatatttgttattgaactaagattaactaaatactttcttttattttccttttttttgacaatcataattgttgtctatttgtgcttaattcagcttttaaagaagggcgattaaccagattaattttaattttcataatcttcttcttctcctgctgttactttcaaaatcttgggcgcttcatttgctgttattttatgagtttatgtatattaaagctacgaagtattgctttctgtttgttttacattactaacttgctactcttgtatcttattcagaagacctttcatcttttctttagtgttgtctagctacgtatattttttttgttaggtcttcttatctgtttcttataattatagtatctttttttctgagaataaatgtttt

SEQ ID NO: 6 - protein, 392 aa, Rhizopus arrhizus
MVSFISISQGVSLCLLVSSMMLGSSAVPVSGKSGSSTTAVSASDNSALPPLISSRCAPPSNKGSKSDLQAEPYYMQKNTEWYESHGGNLTSIGKRDDNLVGGMTLDLPSDAPPISLSGSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTANPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSLLDHLSYFDINEGSCL

SEQ ID NO: 7 - DNA, 942 bp, Nitrosomonas europaea
  CDS (1)...(942)
atggagtcgaaaaatgagcctggggcgtccgccttactgcgtgtccttacgctggacggcggcggcgcgaagggcttttacacgctgggtgtactcaaggaaatcgaggcgatggtcgggtgccctttgcaccagaagtttgatctggttttcggtaccagtacgggcgcgatcatcgcgtcactgatcgcgctcggccacagcgtcgattccatcctggagctgtaccgcaagcacgtgcctaccgtgatgtcgcagaaaaccgctccggccaggtcgcaggccttgaagaagctagctagcgaggtcttcggcgatgcaacgttcagtgatgtgaagaccggcatcgggatcgtcacggccaagtggctgaccgagcgcccaatgatcttcaagggcagcgtcgcgcaggcgcacggccaagtcggcacgttcgtcccgggctttggcgtgagcatcgcagacgccgtcaaggcatcgtgctcggcctacccgttcttcgagcgaacggtagtgaggacttcaatgggcgaggacatcgagctaattgacggcgggtactgtgcaaacaacccgactttgtacgcgatcgccgatgcggttcaggcgcttcggagtgatcgcaaggacatccggctggtgagcgtcggcgtgggcatctaccccgacccgaagccgagcctgctgatgtggttggcgaagaaatatctcgtcagcgtccagttgctgcagaagaccctggagatcaacacgcagtcgatggaccagctgcggcagattctgttccctgacttgctgaccatccgtatcaacgactcctacgtcacgcctgaaatggcgaccgatctgctggagcacgacctcaagaagctgggcatcttgttccagcgaggacgggagtccttcgcgtcgcgtgagaagcaacttcgcgagtatttgata

SEQ ID NO: 8 - protein, 314 aa, Nitrosomonas europaea
MESKNEPGASALLRVLTLDGGGAKGFYTLGVLKEIEAMVGCPLHQKFDLVFGTSTGAIIASLIALGHSVDSILELYRKHVPTVMSQKTAPARSQALKKLASEVFGDATFSDVKTGIGIVTAKWLTERPMIFKGSVAQAHGQVGTFVPGFGVSIADAVKASCSAYPFFERTVVRTSMGEDIELIDGGYCANNPTLYAIADAVQALRSDRKDIRLVSVGVGIYPDPKPSLLMWLAKKYLVSVQLLQKTLEINTQSMDQLRQILFPDLLTIRINDSYVTPEMATDLLEHDLKKLGILFQRGRESFASREKQLREYLI

SEQ ID NO: 9 - DNA, 1307 bp, Pentaclethra macroloba
  CDS (31)...(1257)
cggcacgagctcgtacagattctatccattatgaagtcgaaaatggccatgctccttttgttattttgtgtgttatctaatcagctagtggcagcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattctgatacgaattgaagttgtttcctccttgccatatagcctcactttgtttggc

SEQ ID NO: 10 - protein, 408 aa, Pentaclethra macroloba
MKSKMAMLLLLFCVLSNQLVAAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 11 - DNA, 1227 bp, Artificial Sequence
  Lipase (codon optimized mopentin)
atgaagtccaagatggccatgctcctcctcctcttctgcgtgctctccaaccagctcgtggccgcgttctccacccaggccaaggcctccaaggacggcaacctcgtgaccgtgctcgccatcgacggcggcggcatccgcggcatcatcccgggcgtgatcctcaagcagctcgaggcgaccctccagaggtgggactccagcgccaggctcgcggagtacttcgacgtggtggccggcacctccaccggcggcatcatcaccgccatcctcaccgccccggacccgcagaacaaggaccgcccgctctacgccgccgaggagatcatcgacttctacatcgagcacggcccgtccatcttcaacaagtccaccgcctgctccctcccgggcatcttctgcccgaagtacgacggcaagtacctccaggagatcatctcccagaagctcaacgagaccctcctcgaccagaccaccaccaacgtggtgatcccgtccttcgacatcaagctcctccgcccgaccatcttctccaccttcaagctcgaggaggtgccggagctcaacgtgaagctctccgacgtgtgcatgggcacctccgccgccccgatcgtgttcccgccgtactacttcaagcacggcgacaccgagttcaacctcgtcgacggcgcgatcatcgcggacatcccagccccggtggccctctccgaggtgctccagcaggagaagtacaagaacaaggagatcctcctcctgagcatcggcaccggcgtggtgaagccgggcgagggctactccgccaaccgcacctggaccatcttcgactggtcctccgagaccctcatcggcctcatggggcacggcacccgcgccatgtccgactactacgtgggctcccacttcaaggccctccagccgcagaacaactacctccgcatccaggagtacgacctcgacccggccctcgagtccatcgacgacgcctccaccgagaacatggagaacctcgagaaggtgggccagtccctcctcaacgagccggtgaagcgcatgaacctcaacacgttcgtcgtggaggagaccggcgaggggaccaacgccgaggcgctcgaccgcctcgcccagatcctctacgaggagaagatcacccgcggcctcggcaagatctccctcgaggtggacaacatcgacccgtacaccgagcgcgtgcgcaagctcctcttctga

SEQ ID NO: 12 - protein, 408 aa, Artificial Sequence; identical to SEQ ID NO: 10
  Lipase (codon optimized mopentin)

SEQ ID NO: 13 - DNA, 1404 bp, Solanum tuberosum
  CDS (25)...(1185)
gaaaacactttgaacatttgcaaaatggcaactactaaatcttttttaattttattttttatgatattagcaactactagttcaacatgtgctaagttggaagaaatggttactgtcctaagtattgatggaggtggaattaagggaatcattccagctatcattctcgaatttcttgaaggacaacttcaggaagtggacaataataaagatgcaagacttgcagattactttgatgtaattggaggaacaagtacaggaggtttattgactgctatgataactactccaaatgaaaacaatcgaccctttgctgctgccaaagatattgtacccttttacttcgaacatggccctcatatttttaattatagtggttcaatttttggcccaaggtatgatggaaaatatcttctgcaagttcttcaagaaaaacttggagaaactcgtgtgcatcaagctttgacagaagttgccatctcaagctttgacataaaaacaaataagccagtaatattcactaagtcaaatttagcaaagtctccagaattggatgctaagatgtatgacatatgctattccatagcagcagctccaatatattttcctccacatcactttgttactcatactagtaatggtgctacatatgagttcaatcttgttgatggtggtgttgctactgttggtgatccggcgttattatcccttagcgttgcaacgagacttgcacaagaggatccagcattttcttcaattaagtcattggattacaagcaaatgttgttgctctcattaggcactggcactaattcagagtttgataaaacatatacagcagaagaggcagctaaatggggtcctctacgatggatgttagctatacagcaaatgactaatgcagcaagttcttacatgactgattattacatttctactgtttttcaagctcgtcattcacaaaacaattacctcagggttcaagaaaatgcattaacaggcacaactactgaaatggatgatgcgtctgaggctaatatggaattattagtacaagttggtgaaacattattgaagaaaccagtttccaaagacagtcctgaaacctatgaggaagctctaaagaggtttgcaaaattgctctctaataggaagaaactccgagcaaacaaagcgtcttattaattcaaggtctcgggttgtagtggtaaccttactatgctaaataataagcgcttgcaatatttatgattgcacgcatttaagtatttcaaccttcaaaataaaaggagtttgagggataaatttcaatagaaatgtctctctatgtaatgtgtgcttggattatgtaaccttttggttgtgttaaatatttaaataaattatcgttatttatgttcaagt

SEQ ID NO: 14 - protein, 386 aa, Solanum tuberosum
MATTKSFLILFFMILATTSSTCATLGEMVTVLSIDGGGIKGIIPAIILEFLEGQLQEVDNNKDARLADYFDVIGGTSTGGLLTAMITTPNENNRPFAAAKDIVPFYFEHGPHIFNYSGSIFGPRYDGKYLLQVLQEKLGETRVHQALTEVAISSFDIKTNKPVIFTKSNLAESPQLDAKMYDICYSTAAAPIYFPPHHFVTHTSNGATYEFNLVDGAVATVGDPALLSLSVATRLAQDDPAFSSIKSLDYKQMLLLSLGTGTNSEFDKTYTAEEAAKWGPLRWMLAIQQMTNAASSYMTDYYISTVFQARHSQNNYLRVQENALTGTTTEMDDASEANMELLVQVGETLLKKPVSKDSPETYEEALKRFAKLLSDRKKLRANKASH

SEQ ID NO: 15 - DNA, 2022 bp, Bacillus thuringiensis
  misc_feature (0)...(0): Bt1218K03
atgagtccaaataatcaaaatgaatatgaaattatagatgcgacaccttctacttctgtatccaatgattctaacagatacccttttgcgaatgagccaacaaatgcgctacaaaatatggattataaagattatttaaaaatgtctgcgggaaatgctagtgaataccctggttcacctgaagtacttgttagcggacaagatgcagctaaggccgcaattgatatagtaggtaaattactatcaggtttaggggtcccatttgttgggccgatagtgagtctttatactcaacttattgatattctgtggccttcaggggaaaagagtcaatgggaaatttttatggaacaagtagaagaactcattaatcaaaaaatagcagaatatgcaaggaataaagcgctttcggaattagaaggattaggtaataattaccaattatatctaactgcgcttgaagaatgggaagaaaatccatttcgacgaggttttcgacgaggtgccttacgagatgtgcgaaatcgatttgaaatcctggatagtttatttacgcaatatatgccatcttttagagtgacaaattttgaagtaccattccttactgtatatgcaatggcagccaaccttcatttactgttattaaaggacgcgtcaatttttggagaagaatggggatggtcaacaactactattaataactattatgatcgtcaaatgaaacttactgcagaatattctgatcactgtgtaaagtggtatgaaactggtttagcaaaattaaaaggcacgagcgctaaacaatgggttgactataaccaattccgtagagaaatgacactggcggttttagatgttgttgcattattcccaaattatgacacacgcacgtacccaatggaaacgaaagcacaactaacaagggaagtatatacagatccactgggcgcggtaaacgtgtcttcaattggttcctggtatgacaaagcaccttctttcggagtgatagaatcatccgttattcgaccaccccatgtatttgattatataacgggactcacagtgtatacacaatcaagaagcatttcttccgctcgctatataagacattgggctggtcatcaaataagctaccatcgtgtcagtaggggtagtaatcttcaacaaatgtatggaactaatcaaaatctacacagcactagtacctttgattttacgaattatgatatttacaagactctatcaaaggatgcagtactccttgatattgtttaccctggttatacgtatatattttttggaatgccagaagtcgagtttttcatggtaaaccaattgaataataccagaaagacgttaaagtataatccagtttccaaagatattatagcgagtacaagagattcggaattagaattacctccagaaacttcagatcaaccaaattatgagtcatatagccatagattatgtcatatcacaagtattcccgcgacgggtaacactaccggattagtacctgtattttcttggacacatcgaagtgcagatttaaacaatacaatatattcagataaaatcactcaaattccggccgttaaatgttgggataatttaccgtttgttccagtggtaaaaggaccaggacatacaggaggggatttattacagtataatagaagtactggttctgtaggaaccttatttctagctcgatatggcctagcattagaaaaagcagggaaatatcgtgtaagactgagatatgctactgatgcagatattgtattgcatgtaaacgatgctcagattcagatgccaaaaacaatgaacccaggtgaggatctgacatctaaaacttttaaagttgcagatgctatcacaacagtaaatttagcaacagatagttcggtagcagtgaaacataatttaggtgaagaccctaattcaacattatctggtatagtttacgttgaccgaatcgaattcatcccagtagatgagacatatgaagcggaataa

SEQ ID NO: 16 - protein, 673 aa, Bacillus thuringiensis
MSPNNQNEYEIIDATPSTSVSNDSNRYPFANEPTNALQNMDYKDYLKMSAGNASEYPGSPEVLVSGQDAAKAAIDIVGKLLSGLGVPFVGPIVSLYTQLIDILWPSGEKSQWEIFMEQVEELINQKIAEYARNKALSELEGLGNNYQLYLTALEEWEENPFRRGFRRGALRDVRNRFEILDSLFTQYMPSFRVTNFEVPFLTVYAMAANLHLLLLKDASIFGEEWGWSTTTINNYYDRQMKLTAEYSDHCVKWYETGLAKLKGTSAKQWVDYNQFRREMTLAVLDVVALFPNYDTRTYPMETKAQLTREVYTDPLGAVNVSSIGSWYDKAPSFGVIESSVIRPPHVFDYITGLTVYTQSRSISSARYIRHWAGHQISYHRVSRGSNLQQMYGTNQNLHSTSTFDFTNYDIYKTLSKDAVLLDIVYPGYTYIFFGMPEVEFFMVNQLNNTRKTLKYNPVSKDIIASTRDSELELPPETSDQPNYESYSHRLCHITSIPATGNTTGLVPVFSWTHRSADLNNTIYSDKITQIPAVKCWDNLPFVPVVKGPGHTGGDLLQYNRSTGSVGTLFLARYGLALEKAGKYRVRLRYATDADIVLHVNDAQIQMPKTMNPGEDLTSKTFKVADAITTVNLATDSSVAVKHNLGEDPNSTLSGIVYVDRIEFIPVDETYEAE

SEQ ID NO: 17 - DNA, 2022 bp, Bacillus thuringiensis
  CDS (1)...(2022)
  misc_feature (0)...(0): Bt1218K04
atgagtccaaataatcaaaatgaatatgaaattatagatgcgacaccttctacttctgtatccaatgattctaacagatacccttttgcgaatgagccaacaaatgcgctacaaaatatggattataaagattatttaaaaatgtctgcgggaaatgctagtgaataccctggttcacctgaagtacttgttagcggacaagatgcagctaaggccgcaattgatatagtaggtaaattactatcaggtttaggggtcccatttgttgggccgatagtgagtctttatactcaacttattgatattctgtggccttcaggggaaaagagtcaatgggaaatttttatggaacaagtagaagaactcattaatcaaaaaatagcagaatatgcaaggaataaagcgctttcggaattagaaggattaggtaataattaccaattatatctaactgcgcttgaagaatgggaagaaaatccatttcgacgaggttttcgacgaggtgccttacgagatgtgcgaaatcgatttgaaatcctggatagtttatttacgcaatatatgccatcttttagagtgacaaattttgaagtaccattccttactgtatatgcaatggcagccaaccttcatttactgttattaaaggacgcgtcaatttttggagaagaatggggatggtcaacaactactattaataactattatgatcgtcaaatgaaacttactgcagaatattctgatcactgtgtaaagtggtatgaaactggtttagcaaaattaaaaggcacgagcgctaaacaatgggttgactataaccaattccgtagagaaatgacactggcggttttagatgttgttgcattattcccaaattatgacacacgcacgtacccaatggaaacgaaagcacaactaacaagggaagtatatacagatccactgggcgcggtaaacgtgtcttcaattggttcctggtatgacaaagcaccttctttcggagtgatagaatcatccgttattcgaccaccccatgtatttgattatataacgggactcacagtgtatacacaatcaagaagcatttcttccgctcgctatataagacattgggctggtcatcaaataagctaccatcgtgtcagtaggggtagtaatcttcaacaaatgtatggaactaatcaaaatctacacagcactagtacctttgattttacgaattatgatatttacaagactctatcaaaggatgcagtactccttgatattgtttaccctggttatacgtatatattttttggaatgccagaagtcgagtttttcatggtaaaccaattgaataataccagaaagacgttaaagtataatccagtttccaaagatattatagcgagtacaagagattcggaattagaattacctccagaaacttcagatcaaccaaattatgagtcatatagccatagattatgtcatatcacaagtattcccgcgacgggtaacactaccggattagtacctgtattttcttggacacatcgaagtgcagatttaaacaatacaatatattcagataaaatcactcaaattccggccgttaaatgttgggataatttaccgtttgttccagtggtaaaaggaccaggacatacaggaggggatttattacagtataatagaagtactggttctgtaggaaccttatttctagctcgatatggcctagcattagaaaaagcagggaaatatcgtgtaagactgagatatgctactgatgcagatattgtattgcatgtaaacgatgctcagattcagatgccaaaaacaatgaacccaggtgaggatctgacatctaaaacttttaaagttgcagatgctatcacaacagtaaatttagcaacagatagttcggtagcagtgaaacataatgtaggtgaagaccctaattcaacattatctggtatagtttacgttgaccgaatcgaattcatcccagtagatgagacatatgaagcggaataa

SEQ ID NO: 18 - protein, 673 aa, Bacillus thuringiensis
MSPNNQNEYEIIDATPSTSVSNDSNRYPFANEPTNALQNMDYKDYLKMSAGNASEYPGSPEVLVSGQDAAKAAIDIVGKLLSGLGVPFVGPIVSLYTQLIDILWPSGEKSQWEIFMEQVEELINQKIAEYARNKALSELEGLGNNYQLYLTALEEWEENPFRRGFRRGALRDVRNRFEILDSLFTQYMPSFRVTNFEVPFLTVYAMAANLHLLLLKDASIFGEEWGWSTTTINNYYDRQMKLTAEYSDHCVKWYETGLAKLKGTSAKQWVDYNQFRREMTLAVLDVVALFPNYDTRTYPMETKAQLTREVYTDPLGAVNVSSIGSWYDKAPSFGVIESSVIRPPHVFDYITGLTVYTQSRSISSARYIRHWAGHQISYHRVSRGSNLQQMYGTNQNLHSTSTFDFTNYDIYKTLSKDAVLLDIVYPGYTYIFFGMPEVEFFMVNQLNNTRKTLKYNPVSKDIIASTRDSELELPPETSDQPNYESYSHRLCHITSIPATGNTTGLVPVFSWTHRSADLNNTIYSDKITQIPAVKCWDNLPFVPVVKGPGHTGGDLLQYNRSTGSVGTLFLARYGLALEKAGKYRVRLRYATDADIVLHVNDAQIQMPKTMNPGEDLTSKTFKVADAITTVNLATDSSVAVKHNVGEDPNSTLSGIVYVDRIEFIPVDETYEAE

SEQ ID NO: 19 - DNA, 2022 bp, Bacillus thuringiensis
  CDS (1)...(2022)
  misc_feature (0)...(0): Bt1218-1K054B
atgagcccaaacaaccagaacgagtacgagatcatcgacgccaccccaagcaccagcgtgagcaacgacagcaaccgctacccattcgccaacgagccaaccaacgccctccagaacatggactacaaggactacctgaagatgagcgccggcaacgccagcgagtacccaggcagcccagaggtgctggtgagcggccaggacgccgccaaggccgccatcgacatcgtgggcaagctgctgagcggcctgggcgtgccattcgttggcccaatcgtgagcctgtacacccagctgatcgacatcctgtggccaagcggcgagaagagccagtgggagatcttcatggagcaggtggaggagctgatcaaccagaagatcgccgagtacgccaggaacaaggccctgagcgagctcgagggcctgggcaacaactaccagctgtacctgaccgccctggaggagtgggaggagaacccattcaggaggggcttcaggaggggcgccctgagggacgtgaggaacaggttcgagatcctggacagcctgttcacccagtacatgcctagcttcagggtgaccaacttcgaggtgccattcctgaccgtgtacgctatggccgccaacctgcacctgctgctgctgaaggacgccagcatcttcggcgaggagtggggctggagcaccaccaccatcaacaactactacgacaggcagatgaagctgaccgccgagtacagcgaccactgcgtgaagtggtacgagaccggcctggccaagctgaagggcaccagcgccaagcagtgggtggactacaaccagttcaggagggagatgaccctggccgtgctggacgtggtggccctgttcccaaactacgacaccaggacctacccaatggagaccaaggcccagctgaccagggaggtgtacaccgacccactgggcgccgtgaacgtgagcagcatcggcagctggtacgacaaggccccaagcttcggcgtgatcgagagcagcgtgatcaggccaccacacgtgttcgactacatcaccggcctgaccgtgtacacccagagcaggagcatcagcagcgccagatacatcaggcactgggccggccaccagatcagctaccacagggtgagcaggggcagcaacctgcagcagatgtacggcaccaaccagaacctgcacagcaccagcaccttcgacttcaccaactacgacatctacaagaccctgagcaaggacgccgtgctgctggacatcgtgtacccaggctacacctacatcttcttcggcatgccagaggtggagttcttcatggtgaaccagctgaacaacaccaggaagaccctgaagtacaacccagtgagcaaggacatcatcgcttctacaagagattctgagcttgagcttccaccagagacatctgatcagccaaattacgagtcttactctcatagactttgccatattacatctattccagctacaggcaatactacaggtcttgttccagttttctcttggacacatagatctgctgatcttaataatactatctactctgataagattacacagattccagctgttaagtgctgggataatcttccattcgttccagttgttaagggcccaggtcatacaggtggtgatcttcttcagtacaatagatctacaggttctgttggtacacttttccttgctagatacggtcttgctcttgagaaggctggtaagtacagagttagacttagatacgctacagatgctgatattgttcttcatgttaatgatgctcagattcagatgccaaagacaatgaatccaggcgaggatcttacatctaagacattcaaggttgctgatgctattacaacagttaatcttgctacagattcttctgttgctgttaagcacaatgttggcgaggacccaaattctacactttctggtattgtttacgttgataggattgagttcattccagttgatgagacatacgaggctgagtga

SEQ ID NO: 20 - protein, 673 aa, Bacillus thuringiensis; identical to SEQ ID NO: 18

(19) United States
(12) Patent Application Publication
(10) Pub. No.: US 2005/0188439 A1
(43) Pub. Date: Aug. 25, 2005

(54) Methods for enhancing insect resistance in plants

(75) Inventors: Billy F. McCutchen, Clive, IA (US); Andre R. Abad, W. Des Moines, IA (US)

Correspondence Address: ALSTON & BIRD LLP;PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignees: Pioneer Hi-Bred International, Inc., Des Moines, IA (US); E.I. duPont de Nemours and Company, Wilmington, DE (US)

(21) Appl. No.: 11/061,894

(22) Filed: Feb. 18, 2005

Related U.S. Application Data
(60) Provisional application No. 60/546,533, filed on Feb. 20, 2004.
(60) Provisional application No. 60/546,845, filed on Feb. 23, 2004.

(51) Int. Cl. A01H 1/00; C12N 15/82

(52) U.S. Cl. 800/279; 435/468

(57) ABSTRACT

Methods for creating and enhancing insect resistance in plants are provided. The methods comprise stably introducing into a plant a combination of polynucleotides comprising a sequence encoding a lipase polypeptide having insecticidal activity and a sequence encoding a Bt insecticidal protein, where each of these coding sequences is operably linked to a promoter that drives expression in a plant cell. Plants with enhanced insect resistance and seed thereof are also provided. The methods of the invention may be used in a variety of agricultural systems for controlling insect pests, including propagating lineages of insect-resistant crops and targeting coexpression of insecticidal lipase and Bt insecticidal protein to plant organs that are particularly susceptible to infestation.

### CROSS-REFERENCE TO RELATED APPLICATIONS

This application claims the benefit of U.S. Provisional Application Ser. No. 60/546,533, filed Feb. 20, 2004, and U.S. Provisional Application Ser. No. 60/546,845, filed Feb. 23, 2004, the contents of both of which are herein incorporated by reference in their entirety.

### FIELD OF THE INVENTION

The present invention relates to the field of plant molecular biology and insect pest control, particularly to methods for controlling insect species via coexpression of insecticidal proteins having different modes of action.

### BACKGROUND OF THE INVENTION

Insect pests are a serious problem in agriculture. They destroy millions of acres of staple crops such as corn, soybeans, peas, and cotton. Yearly, these pests cause over $100 billion dollars in crop damage in the U.S. alone. In an ongoing seasonal battle, farmers must apply billions of gallons of synthetic pesticides to combat these pests. However, synthetic pesticides pose many problems. They are expensive, costing U.S. farmers almost $8 billion dollars per year. They force the emergence of insecticide-resistant pests, and they can harm the environment.

Other approaches to pest control have been tried. In some cases, crop growers have introduced “natural predators” of the species sought to be controlled, such as non-native insects, fungi, and bacteria like Bacillus thuringiensis. Alternatively, crop growers have introduced large colonies of sterile insect pests in the hope that mating between the sterilized insects and fecund wild insects would decrease the insect population. Unfortunately, success has been equivocal and the expense considerable. For example, as a practical matter, introduced species rarely remain on the treated land-spreading to other areas as an unintended consequence. Predator insects migrate, and fungi or bacteria wash off of plants into streams and rivers. Consequently, crop growers need more practical and effective solutions.

Certain species of microorganisms of the genus Bacillus are known to possess pesticidal activity against a broad range of insect pests including Lepidoptera, Diptera, Coleoptera, Hemiptera, and others. Bacillus thuringiensis and Bacillus papilliae are among the most successful biocontrol agents discovered to date. Insect pathogenicity has been attributed to strains of: B. larvae, B. lentimorbus, B. papilliae, B. sphaericus, B. thuringiensis (Harwook, ed. (1989) Bacillus (Plenum Press), p. 306) and B. cereus (European Patent No. EP0792363). Pesticidal activity appears to be concentrated in parasporal crystalline protein inclusions, although insecticidal proteins have also been isolated from the vegetative growth stage of Bacillus. Several genes encoding these insecticidal proteins have been isolated and characterized (see, for example, U.S. Pat. Nos. 5,366,892 and 5,840,868).

Microbial pesticides, particularly those obtained from Bacillus strains, have played an important role in agriculture as alternatives to chemical pest control. Insecticidal proteins isolated from strains of Bacillus thuringiensis, known as 8-endotoxins or Cry toxins, are initially produced in an inactive protoxin form. These protoxins are proteolytically converted into an active toxin through the action of proteases in the insect gut. See, Rukmini et al. (2000) Biochimie 82:109-116; Oppert (1999) Arch. Insect Biochem. Phys. 42:1-12 and Carroll et al. (1997) J. Invertebrate Pathology 70:41-49. Proteolytic activation of the toxin can include the removal of the N- and C-terminal peptides from the protein, as well as internal cleavage of the protein. Other proteases can degrade insecticidal proteins. See Oppert, ibid.; see also U.S. Pat. Nos. 6,057,491 and 6,339,491. Once activated, the Cry toxin binds with high affinity to receptors on epithelial cells in the insect gut, thereby creating leakage channels in the cell membrane, lysis of the insect gut, and subsequent insect death through starvation and septicemia. See, e.g., Li et al. (1991) Nature 353:815-821.

Recently, agricultural scientists have developed crop plants with enhanced insect resistance by genetically engineering crop plants to produce insecticidal proteins from Bacillus. For example, corn and cotton plants genetically engineered to produce Cry toxins (see, e.g., Aronson (2002) Cell Mol. Life Sci. 59(3):417-425; Schnepf et al. (1998) Microbiol. Mol. Biol. Rev. 62(3):775-806) are now widely used in American agriculture and have provided the farmer with an environmentally friendly alternative to traditional insect-control methods. In addition, potatoes genetically engineered to contain pesticidal Cry toxins have been sold to the American farmer. However, these Bt insecticidal proteins only protect plants from a relatively narrow range of pests. Thus, there is an immediate need for methods that enhance the effects of Bt insecticidal proteins.

### SUMMARY OF THE INVENTION

Methods for creating or enhancing insect resistance in plants are provided. The compositions and methods of the invention may be used in a variety of systems for controlling plant and non-plant pests, including propagating lineages of insect-resistant crops and targeting expression of pesticidal proteins to plant organs that are particularly susceptible to infestation, such as roots and leaves. These methods also find use in insect resistance management.

The methods of the invention comprise genetically modifying a plant to express at least one lipase polypeptide having insecticidal activity in combination with at least one Bacillus thuringiensis (Bt) insecticidal protein. The insecticidal properties of the lipase polypeptide coupled with the second mode of action of the Bt insecticidal protein provide for synergistic control of insect pests. The compositions of the invention further comprise constructs that provide for expression of insecticidal lipases, such as lipid acyl hydrolases, in combination with Bt insecticidal proteins in plants. DNA sequences encoding such lipases and Bt insecticidal proteins useful in the practice of the invention are also provided, including DNA sequences that are optimized for expression in plants. The DNA sequences encoding these insecticidal lipases can be used to transform plants and other organisms for the control of pests. Also provided are transformed plants, plant tissues and cells, and seeds thereof that have been genetically modified using the methods of the present invention to create or enhance their resistance to insect pests.

The compositions and methods of the invention may be used in a variety of agricultural systems for controlling plant and non-plant pests, including propagating lineages of insect-resistant crops and targeting coexpression of insecticidal lipases and Bt insecticidal proteins to plant organs.

### BRIEF DESCRIPTION OF THE DRAWINGS

FIG. 1 shows western corn rootworm (WCRW) bioassay results from feeding pentin (lipase) and Bt toxin to developing larvae. The diet causes a dose-dependent inhibition of larval growth as a percentage of wild-type controls.

### DETAILED DESCRIPTION OF THE INVENTION

The present invention is drawn to methods of creating and enhancing insect resistance in plants by introducing polynucleotides encoding insecticidal lipases and Bacillus thuringiensis (Bt) insecticidal proteins. As will be described herein, these methods are useful for conferring insect resistance to a wide variety of plants, including crops and other domesticated plant species.

In particular embodiments, the methods of the invention comprise stably introducing a combination of polynucleotides into plants, the combination comprising at least one polynucleotide comprising a sequence encoding an insecticidal lipase and at least one polynucleotide comprising a sequence encoding a Bt insecticidal protein, each of which is operably linked to a promoter that drives expression in a plant cell. “Stably introducing” is intended to mean that the introduced nucleotide sequences are integrated into the genome of the plant. Once the combination of polynucleotides is introduced into the cells of the plant, the encoded insecticidal lipase and Bt insecticidal protein are transcribed and translated by the endogenous cellular machinery. When insects attempt to feed or lay eggs in the transgenic plant, the combined expression of the insecticidal lipase and Bt insecticidal protein kills the insects or inhibits their growth. Thus, plant cells, organs, seeds, and/or the entire plant are thereby made resistant to infestation. Because the cells are stably transformed by these methods, the invention is useful in creating seed and filial lines that are also insect resistant.

Additionally, it has been unexpectedly found that the coexpression of Bt insecticidal protein and lipase transgenes creates a synergistic insecticidal effect. This effect is useful in decreasing the required effective dose. Synergy also decreases the effective amount of insecticidal protein that a plant must produce, thereby lessening the carbon/nitrogen load associated with plant defense and increasing the effective yield of the plant. Thus, many beneficial properties are conferred on a transgenic plant expressing a combination of these two classes of insecticidal proteins, i.e., a Bt insecticidal protein and a lipase polypeptide having insecticidal activity (hereinafter referred to as “insecticidal lipases”).

Insecticidal lipases, such as lipid acyl hydrolases, and Bt insecticidal proteins that can impact an insect pest find use in practicing the methods of the invention. The term “impact an insect pest” or “impacting an insect pest” is intended to mean the effect of employing any substance or organism to prevent, destroy, repel, or mitigate an insect pest. Thus, many beneficial properties are conferred on a transgenic plant expressing insecticidal proteins, e.g., lipase polypeptides and Bt polypeptides having pesticidal activity.

As used herein, the term “pesticidal activity” is used to refer to activity of an organism or a substance (such as, for example, a protein), whether toxic or inhibitory, that can be measured by, but is not limited to, pest mortality, pest weight loss, pest repellency, pest growth stunting, and other behavioral and physical changes of a pest after feeding and exposure for an appropriate length of time. In this manner, pesticidal activity impacts at least one measurable parameter of pest fitness. Similarly, “insecticidal activity” may be used to refer to “pesticidal activity” when the pest is an insect pest. “Stunting” is intended to mean greater than 50% inhibition of growth as determined by weight. General procedures for monitoring insecticidal activity include addition of the experimental compound or organism to the diet source in an enclosed container. Assays for assessing insecticidal activity are well known in the art. See, e.g., U.S. Pat. Nos. 6,570,005 and 6,339,144; herein incorporated by reference in their entirety. The optimal developmental stage for testing for insecticidal activity is larvae or immature forms of an insect of interest. The insects may be reared in total darkness at from about 20° C. to about 30° C. and from about 30% to about 70% relative humidity. Bioassays may be performed as described in Czapla and Lang (1990) J. Econ. Entomol. 83(6):2480-2485. Methods of rearing insect larvae and performing bioassays are well known to one of ordinary skill in the art.

The term “pesticidally effective amount” connotes a quantity of a substance or organism that has pesticidal activity when present in the environment of a pest. For each substance or organism, the pesticidally effective amount is determined empirically for each pest affected in a specific environment. Similarly, an “insecticidally effective amount” may be used to refer to a “pesticidally effective amount” when the pest is an insect pest. “Creating or enhancing insect resistance” is intended to mean the plant genetically modified in accordance with the methods of the present invention has increased resistance to one or more insect pests relative to a plant having a similar genetic component with the exception of the genetic modification described herein. Genetically modified plants of the present invention are capable of expression of at least one insecticidal lipase and at least one Bt insecticidal protein, the combination of which protects a plant from an insect pest while impacting an insect pest of a plant. “Protects a plant from an insect pest” is intended to mean the limiting or eliminating of insect pest-related damage to a plant by, for example, inhibiting the ability of the insect pest to grow, feed, and/or reproduce or by killing the insect pest. As used herein, “impacting an insect pest of a plant” includes, but is not limited to, deterring the insect pest from feeding further on the plant, harming the insect pest by, for example, inhibiting the ability of the insect to grow, feed, and/or reproduce, or killing the insect pest.

Toxic and inhibitory effects of insecticidal lipases and Bt proteins include, but are not limited to, stunting of larval growth, killing eggs or larvae, reducing either adult or juvenile feeding on transgenic plants relative to that observed on wild-type, and inducing avoidance behavior in an insect as it relates to feeding, nesting, or breeding. The term “insecticidal lipase” is used in its broadest sense and includes, but is not limited to, any member of the family of lipid acyl hydrolases that has toxic or inhibitory effects on insects. Also, the term “Bt insecticidal protein” is used in its broadest sense and includes, but is not limited to, any member of the family of Bt proteins that have toxic or inhibitory effects on insects, such as Bt toxins described herein and known in the art. Thus, as described herein, insect resistance can be conferred to an organism by introducing a nucleotide sequence encoding an insecticidal lipase with a sequence encoding a Bt insecticidal protein or applying an insecticidal substance, which includes, but is not limited to, an insecticidal protein, to an organism (e.g., a plant or plant part thereof).

Insecticidal lipases expressed in combination with Bt insecticidal proteins find use as an alternative to a previously implemented pesticide method such as pesticide application and/or prior genetic modification of a plant. Measures aimed at reducing the potential for insect pests to become resistant to a pesticide are termed “insect resistance management.” Insecticidal lipases, such as insecticidal lipid acyl hydrolases including but not limited to those disclosed herein, as well as Bt insecticidal proteins disclosed herein and known in the art are of use in such insect resistance management programs because they can be used as an alternative to current pesticides.

Therefore, the insecticidal lipases and Bt insecticidal proteins that find use in the invention may also be further selected for use in insect resistance management programs. Those of skill in the art recognize that selection of a particular insecticidal lipase, such as a lipid acyl hydrolase, and/or a particular Bt insecticidal protein will depend on the type of resistant insect strain that emerges (or is likely to emerge) as well as the crops that are likely to suffer from infestation.

Any nucleotide sequence encoding a lipase polypeptide that has insecticidal activity can be used to practice the methods of the invention. The term “insecticidal lipase” includes any member of the family of lipid acyl hydrolases that has toxic or inhibitory effects on insects. Lipases are well known in the art. One class of lipases is the lipid acyl hydrolase class, also known as triacylglycerol acylhydrolases or triacylglycerol lipases (termed EC 3.1.1.3 enzymes under the IUBMB nomenclature system). These enzymes catalyze the hydrolysis reaction: triacylglycerol+H₂O=diacylglycerol+a carboxylate. Lipid acyl hydrolases all share a common, conserved scissile structural region termed the catalytic triad. The catalytic triad consists of a glycine-X amino acid-serine-X amino acid-glycine motif (GxSxG). It has been demonstrated that amino acid substitution in this region abrogates enzymatic activity. Remarkably, the enzymatic action of these lipid acyl hydrolases also correlates with significant insecticidal activity. See, for example, the insecticidal lipases disclosed in copending U.S. Non-provisional application Ser. No. ______ filed Feb. ______, 2005 (Attorney Docket No. 035718/286812) which claims the benefit of U.S. Provisional Application Ser. No. 60/546,605, entitled “Lipases and Methods of Use,” filed Feb. 20, 2004; herein incorporated by reference.

The combination of polynucleotides to be introduced into a plant can comprise a coding sequence for one or more insecticidal lipases. Insecticidal lipases may be derived from plants and non-plants. “Non-plant” is intended to mean encompassing all of the phylogentic Kingdoms except Planta (i.e., encompasssing Kingdom Eubacteria, Kingdom Euryarcheota, Kingdom Crenarcheota, Kingdom Protozoa, Kingdom Mycota, Kingdom Chromista, and Kingdom Animalia). Examples of non-plant lipase sequences useful for the practice of the present invention include Candida lipase 1 (CLIP1) derived from the yeast Candida cylindracea (previously known as Candida rugosa) (NCBI Accession No. X16712) (see, for example, SEQ ID NO:1, encoding SEQ ID NO:2); lipase derived from Rhizopus arrhizus (NCBI Accession No. AF229435) (see, for example, SEQ ID NO:5, encoding SEQ ID NO:6); lipase derived from Nitrosomonas europaea (e.g., GenBank Accession No. BX321865; nucleotide region 4475-5422 encoding a protein having GenPept Accession No. CAD86430 and deposited as ATCC Accession No. 19718D, and see, for example, SEQ ID NO:7, encoding SEQ ID NO:8); and lipase derived from porcine pancreas (see, for example, SEQ ID NO:4 as encoded by the maize-optimized coding sequence shown in SEQ ID NO:3). Plant lipases of use in practicing the methods of the invention include, but are not limited to, pentin-1 lipase derived from the oil bean tree (see, for example, SEQ ID NO:9, encoding SEQ ID NO:10, and pentin-1 nucleotide sequence optimized for enhanced expression, for example, SEQ ID NO:11, encoding SEQ ID NO:12); (see also, U.S. Pat. Nos. 5,981,722, and 6,339,144, herein incorporated by reference in their entirety); patatin lipase (see U.S. Pat. No. 5,743,477, herein incorporated by reference in its entirety); (see also, for example, SEQ ID NO:13, encoding SEQ ID NO:14); and functional variants or fragments thereof. See also, Longhi et al. (1992) Biochim. Biophys. Acta 1131(2):227-232, and Lotti et al. (1993) Gene 124(1):45-55.

In some embodiments, the methods of the present invention provide for the introduction of one or more lipase-encoding nucleotide sequences comprising sequences set forth in SEQ ID NOs:1, 3, 5, 7, 9, 11, and 13. These sequences encode the insecticidal lipase polypeptides set forth in SEQ ID NOs:2, 4, 6, 8, 10, 12, and 14, respectively. It is recognized that variants and fragments of these coding sequences can be used so long as they encode a lipase polypeptide having insecticidal activity.

In accordance with the methods of the present invention, at least one polynucleotide comprising a sequence encoding a Bt insecticidal protein is introduced into a plant in combination with the introduction of at least one polynucleotide comprising a coding sequence for a lipase polypeptide having insecticidal activity. Any coding sequence for a Bt insecticidal protein can be used. “Bt insecticidal protein” is intended to mean the broader class of toxins found in various strains of Bacillus thuringiensis, which includes such toxins as, for example, the vegetative insecticidal proteins and the δ-endotoxins or cry toxins. The vegetative insecticidal proteins (for example, members of the VIP1, VIP2, or VIP3 classes) are secreted insecticidal proteins that undergo proteolytic processing by midgut insect fluids. They have pesticidal activity against a broad spectrum of Lepidopteran insects. See, for example, U.S. Pat. No. 5,877,012, herein incorporated by reference in its entirety.

The Bt δ-endotoxins are synthesized as protoxins and crystallize as parasporal inclusions. When ingested by an insect pest, the microcrystal structure is dissolved by the alkaline pH of the insect midgut, and the protoxin is cleaved by insect gut proteases to generate the active toxin. The activated Bt toxin binds to receptors in the gut epithelium of the insect, causing membrane lesions and associated swelling and lysis of the insect gut. Insect death results from starvation and septicemia. See, e.g., Li et al. (1991) Nature 353:815-821; Aronson (2002) Cell Mol. Life Sci. 59(3):417-425; Schnepf et al. (1998) Microbiol Mol. Biol. Rev. 62(3):715-806. The Bt δ-endotoxins are toxic to larvae of a number of insect pests, including members of the Lepidoptera, Diptera, and Coleoptera orders. The effectiveness of the Bt insecticidal proteins as a toxin depends on the structure of the toxin, the amount ingested, and the species of larvae ingesting the toxin.

The Bt δ-endotoxins or cry toxins are well known in the art (see, U.S. Patent Application Publication No. 2003/0177528, herein incorporated by reference in its entirety). There are currently over 250 known species of Bt δ-endotoxins with a wide range of specificities and toxicities. See for example, Bacillus thuringiensis toxic proteins described in U.S. Pat. Nos. 5,366,892; 5,747,450; 5,737,514; 5,723,756; 5,593,881; and Geiser et al. (1986) Gene 48:109. For an expansive list see Crickmore et al. (1998) Microbiol. Mol. Biol. Rev. 62:807-813, and for regular updates see Crickmore et al. (2003) “Bacillus thuringiensis toxin nomenclature,” at biols.susx.ac.uk/Home/Neil_Crickmore/Bt/index, which can be accessed on the world-wide web using the “www” prefix. The criteria for inclusion in this list is that the proteins have significant sequence similarity to one or more toxins within the nomenclature or be a Bacillus thuringiensis parasporal inclusion protein that exhibits pesticidal activity, or that it have some experimentally verifiable toxic effect to a target organism.

The δ-endotoxins are related to various degrees by similarities in their amino acid sequences and tertiary structure and means for obtaining the crystal structures of B. thuringiensis endotoxins are well known. Exemplary high-resolution crystal structure solution of both the Cry3A and Cry3B polypeptides are available in the literature. The solved structure of the Cry3A gene (Li et al. (1991) Nature 353:815-821) provides insight into the relationship between structure and function of the endotoxin. A combined consideration of the published structural analyses of B. thuringiensis endotoxins and the reported function associated with particular structures, motifs, and the like indicates that specific regions of the endotoxin are correlated with particular functions and discrete steps of the mode of action of the protein. For example, δ-endotoxins isolated from B. thuringiensis are generally described as comprising three domains: a seven-helix bundle that is involved in pore formation, a three-sheet domain that has been implicated in receptor binding, and a beta-sandwich motif (Li et al. (1991) Nature 305: 815-821).

Coexpression of any Bt δ-endotoxin (whether a holotoxin or insecticidal fragment) is useful for practicing this invention, including all species of Cry and Cyt designated Bt δ-endotoxins. These toxins include Cry 1 through Cry 42, Cyt 1 and 2, Cyt-like toxin, and the binary Bt toxins. In the case of binary Bt toxins, those skilled in the art recognize that two Bt toxins must be co-expressed to induce Bt insecticidal activity. In some embodiments, the methods of the present invention provide for the introduction of a nucleotide sequence encoding a Bt δ-endotoxin selected from the group consisting of Cry 1, Cry 3, Cry 5, Cry 8, and Cry 9. Of particular interest are the Cry 8 or Cry 8-like δ-endotoxins. “Cry 8-like” is intended to mean that the nucleotide or amino acid sequence shares a high degree of sequence identity or similarity to previously described sequences categorized as Cry 8, which includes such toxins as, for example, Cry8Bb1 (see Genbank Accession No. CAD57542) and Cry8Bc1 (see Genbank Accession No. CAD57543). See copending U.S. Patent Application Publication No. 2004/0210963, filed Jun. 25, 2003, herein incorporated by reference in its entirety. “Cry 8-like insect protoxin” is intended to mean to mean the biologically inactive polypeptide that is converted to the activated Cry 8-like insect toxin upon cleavage at a proteolytic activation site by a protease. It is the activated Cry 8-like insect toxin that has pesticidal activity. As used herein, “Cry 8-like insect toxin” refers to a biologically active pesticidal polypeptide that shares a high degree of sequence identity or similarity to Cry 8 insect toxin sequences.

In other embodiments, the methods of the present invention provide for the introduction of at least one Bt insecticidal protein, such as a Bt toxin-encoding nucleotide sequence selected from the group consisting of the sequences set forth in SEQ ID NOs:15 (Bt 1218K03), 17 (Bt 1218K04), and 19 (Bt 1218-1K054B). These sequences encode the Bt 8-endotoxin polypeptides set forth in SEQ ID NOs:16 (Bt 1218K03), 18 (Bt 1218K04), and 20 (Bt 1218-1K054B), respectively. It is recognized that variants and fragments of these coding sequences can be used so long as the encoded Bt toxin polypeptide has insecticidal activity.

The Bt insecticidal proteins to be coexpressed with at least one insecticidal lipase can be naturally occurring proteins, or can be genetically modified forms thereof that provide for improved insecticidal properties. Examples of genetically modified Bt insecticidal proteins include the mutant forms of Cry 8-like insect protoxins having a mutation that comprises an additional, or an alternative, protease-sensitive site that is readily recognized and/or cleaved by a category of proteases, such as mammalian proteases or insect proteases. The presence of an additional and/or alternative protease-sensitive site in the amino acid sequence of the encoded polypeptide can improve the pesticidal activity and/or specificity of the polypeptide compared to that of an unmodified wild-type δ-endotoxin. See, for example, the mutant forms of Cry 8-like insect protoxins disclosed in copending U.S. Patent Application Publication No. 2004/0210963, filed Jun. 25, 2003, entitled “Genes Encoding Proteins with Pesticidal Activity”; herein incorporated by reference in its entirety.

In other embodiments, the Bt insecticidal protein is a Bt δ-endotoxin that has been genetically modified to protect the endotoxin from proteolytic inactivation by plant proteases. In this manner, a proteolytic site within a Bt δ-endotoxin that is susceptible to cleavage by a plant protease is mutated to comprise a site that is not sensitive to the plant protease, thereby protecting the protein from proteolytic inactivation by a plant protease. Such proteolytic protection enhances the stability of the active toxin in a transgenic plant and improves the associated pest resistance properties. See copending U.S. patent application Ser. No. 10/746,914, filed Dec. 24, 2003, entitled “Genes Encoding Proteins with Pesticidal Activity”; herein incorporated by reference in its entirety.

In alternative embodiments, the Bt insecticidal protein is a Bt δ-endotoxin that has been genetically modified for improved proteolytic processing into the activated form of the protoxin. In this manner, the Bt δ-endotoxin is modified to comprise at least one proteolytic activation site that is not naturally occurring within the insect protoxin, and which has been engineered to comprise a cleavage site that either is sensitive to cleavage by a plant protease residing within the cells of a plant, or is sensitive to cleavage by an insect gut protease. “Sensitive to cleavage” is intended to mean that the protease recognizes the cleavage site, and thus is capable of cleaving the protoxin at that cleavage site. In both instances, the non-naturally occurring proteolytic activation site is engineered within an activation region of the insect protoxin. “Activation region” is intended to mean to mean a region within the insect protoxin wherein proteolytic cleavage at the engineered activation site results in the production of a biologically active insect toxin (i.e., the activated form of the insect protoxin). Proteolytic cleavage by the plant protease or insect gut protease releases the activated insect toxin within a plant cell or within the insect gut, respectively. See copending U.S. Patent Application Ser. No. 60/532,185, filed Dec. 23, 2003, entitled “Plant Activation of Insect Toxin”; herein incorporated by reference in its entirety.

Thus, the methods of the present invention comprise introducing a combination of polynucleotides into a plant, where the combination comprises at least one polynucleotide comprising a sequence encoding an insecticidal lipase, such as a lipid acyl hydrolase, and at least one polynucleotide comprising a sequence encoding a Bt insecticidal protein, where each of these coding sequences is operably linked to at least one promoter that drives expression in a plant cell. Those skilled in the art recognize that coexpression of transgenes can create subtractive, additive, or synergistic phenotypic effects. Subtractive effects occur where a second event (e.g., transformation with a second gene or crossbreeding with a second plant expressing a gene of interest) decreases the insecticidal effectiveness relative to the first event. Synergistic effects occur where the action of two or more agents working together produce an effect greater than the combined effect of the same agents used separately; see for example, McCutchen et al. (1997) J. Econ. Entomol. 90:1170-1180; Preisler et al. (1999) J. Econ. Entomol. 92:598-603. Additive effects occur where two or more agents working together produce an effect at least equal to the combined effect of the same agents used separately. In some embodiments of the invention, coexpression of the combination of polynucleotides comprising sequences encoding the insecticidal lipase, such as a lipid acyl hydrolase, and the Bt insecticidal protein provides a synergistic effect, i.e., synergistic enhancement of resistance to one or more insect pests.

The combination of polynucleotides for practicing the present invention may comprise full-length nucleotide sequences encoding the insecticidal lipase or Bt insecticidal protein as well as fragments of the full-length coding sequences, wherein polypeptide fragments are encoded. The term “fragment” is intended to mean a portion of the polynucleotide or a portion of the amino acid sequence and hence protein encoded thereby. Fragments of a polynucleotide may encode protein fragments that retain the biological activity of the native protein and hence retain insecticidal activity. Where the combination of polynucleotides to be introduced into a plant comprises fragments of respective coding sequences, the fragments will likewise encode protein fragments that retain the biological activity of the native protein and hence retain insecticidal activity.

Thus, a fragment of a polynucleotide may encode a biologically active portion of an insecticidal lipase, such as a fragment of a lipid acyl hydrolase. A biologically active portion of a lipase, such as a biologically active portion of a lipid acyl hydrolase, can be prepared by isolating a portion of one of the polynucleotides encoding a lipase, expressing the encoded portion of the lipase protein (e.g., by recombinant expression in vitro), and assessing the activity of the expressed portion of the lipase protein for lipid acyl hydrolase activity. For example, lipid acyl hydrolases retain a conserved amino acid sequence termed the catalytic triad (i.e., GxSxG) as discussed supra. Thus, a fragment of a lipid acyl hydrolase having a catalytic triad finds use in the invention, as it retains enzymatic activity.

A fragment of a polynucleotide that encodes a biologically active portion of an insecticidal lipase useful in the invention, such as a lipid acyl hydrolase, will encode at least 15, 25, 30, 50, 100, 150, 200, 250, or 300 contiguous amino acids, or up to the total number of amino acids present in a full-length lipase protein (for example, 549 amino acids for SEQ ID NO:2, 450 amino acids for SEQ ID NO:4, 392 amino acids for SEQ ID NO:6, and 314 amino acids for SEQ ID NO:8, respectively).

Polynucleotides that are fragments of nucleotide sequences encoding lipases, such as lipid acyl hydrolases, comprise at least 16, 20, 50, 75, 100, 150, 200, 250, 300, 350, 400, 450, 500, 550, 600, 650, 700, 750, 800, 850, 900, or 940 contiguous nucleotides, or up to the number of nucleotides present in a full-length polynucleotide disclosed herein (for example, 1650 nucleotides for SEQ ID NO:1; 1,350 for SEQ ID NO. 3; 3120 nucleotides for SEQ ID NO:5, of which 1178 contiguous nucleotides from 901-2079 are coding sequence; and 942 nucleotides for SEQ ID NO:7).

Additionally, a fragment of a polynucleotide may encode a biologically active portion of a Bt insecticidal protein. Structure-function relationships are well described in the art for Bt insecticidal proteins. The receptor binding domains and the crystal structure are known. See, for example, Carroll et al. (1991) Nature 353:815-821; Hodgman and Ellar (1990) DNA Seq. 1:97-106; Smedley and Ellar (1996) Microbiol. 142:1617-1624; deMaagd et al. (1996) Microbiol. 62:1537-1543; Knight et al. (1994) N. Molec. Microbiol. 11: 429-436; and Carroll et al. (1997) J. Cell Sci. 110:3099-3104.

A fragment of a polynucleotide that encodes a biologically active portion of a Bt insecticidal protein useful in the invention will encode at least 15, 25, 30, 50, 100, 150, 200, 250, 300, 350, or 380 contiguous amino acids, or up to the total number of amino acids present in a full-length Bt insecticidal protein (for example, 408 amino acids for SEQ ID NO:10, 408 amino acids for SEQ ID NO:12, 386 amino acids for SEQ ID NO:14, 673 amino acids for SEQ ID NO:16, 673 amino acids for SEQ ID NO:18, and 673 amino acids for SEQ ID NO:20).

Polynucleotides that are fragments of nucleotide sequences encoding Bt insecticidal proteins comprise at least 16, 20, 50, 75, 100, 150, 200, 250, 300, 350, 400, 450, 500, 550, 600, 650, 700, 750, 800, 850, 900, 950, 1000, 1100, 1200, or 1220 contiguous nucleotides, or up to the number of nucleotides present in a full-length polynucleotide disclosed herein (for example, 1307 nucleotides for SEQ ID NO:9, of which 1226 contiguous nucleotides from 31-1257 are coding sequence; 1227 nucleotides for SEQ ID NO:11; 1404 nucleotides for SEQ ID NO:13; 2022 nucleotides for SEQ ID NO:15; 2022 nucleotides for SEQ ID NO:17; and 2022 nucleotides for SEQ ID NO:19).

A biologically active portion of a Bt insecticidal protein can be prepared by isolating a portion of a full-length coding sequence for the Bt insecticidal protein of interest, expressing the encoded portion of the protein (e.g., by recombinant expression in vitro), and assessing the activity of the encoded portion of the Bt insecticidal protein for insecticidal activity using assays well known in the art.

The methods of the present invention can be practiced using biologically active variants of insecticidal lipases and/or Bt insecticidal proteins. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the lipase polypeptides useful in the invention, such as a lipid acyl hydrolase, or a Bt insecticidal protein. Naturally occurring variants, such as allelic variants, can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an insecticidal lipase protein and/or a Bt insecticidal protein useful in the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to that particular polynucleotide as determined by sequence alignment programs and parameters as described elsewhere herein.

Variants of a particular insecticidal lipase-encoding or Bt insecticidal protein-encoding nucleotide sequence (i.e., the respective reference coding sequence) can also be evaluated by comparison of the percent sequence identity between the lipase polypeptide or Bt insecticidal protein encoded by a variant nucleotide sequence and the lipase polypeptide or Bt insecticidal protein encoded by the reference nucleotide sequence. Thus, for example, isolated nucleic acids that encode an insecticidal lipase polypeptide with a given percent sequence identity to the polypeptide of SEQ ID NO:2, 4, 6, 8, 10, 12, or 14 and/or a Bt insecticidal protein with a given percent sequence identity to the polypeptide of SEQ ID NO:16, 18, or 20 can be utilized in the methods of the invention. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity.

“Variant protein” is intended to mean a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, retaining insecticidal properties and/or lipase activity as described herein. Such variants may result from, for example, genetic polymorphism or from human manipulation. Biologically active variants of a native insecticidal lipase or Bt insecticidal protein will have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the native lipase or Bt insecticidal protein as determined by sequence alignment programs described elsewhere herein using default parameters. A biologically active variant of a lipase or Bt insecticidal protein may differ from a native protein by as few as I-15 amino acid residues, as few as 1-10, such as 6-10, as few as 5, as few as 4, 3, 2, or even 1 amino acid residue.

The encoded insecticidal lipases or Bt insecticidal proteins encompassed by the invention may be altered in various ways including amino acid substitutions, deletions, truncations, and insertions. Methods for such manipulations are generally known in the art. For example, amino acid sequence variants and fragments of lipases, such as lipid acyl hydrolase fragments, and/or Bt insecticidal proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found, Washington, D.C.), herein incorporated by reference. Conservative substitutions, such as exchanging one amino acid with another having similar properties, may be optimal.

Specifically, those of skill in the art will recognize that regions of the nucleotide sequence or amino acid sequence that are highly conserved in lipid acyl hydrolases or Bt insecticidal proteins of the invention when compared to other regions within the sequences, will generally be less tolerant to modification through amino acid substitutions. As such, the previously discussed catalytic triad found in lipid acyl hydrolases, consisting of a glycine-X amino acid-serine-X amino acid-glycine motif (GxSxG), may be preserved in certain embodiments to retain enzymatic and/or biological activity. Likewise, the receptor binding domains and the crystal structure are known for Bt proteins, and as such, the conserved structures may also be preserved in certain embodiments to retain biological activity.

Thus, the nucleotide sequences for use in practicing the invention include both the naturally occurring sequences as well as mutant forms. Likewise, the insecticidal proteins of the invention encompass both naturally occurring proteins as well as variations and modified forms thereof Such variants will continue to possess the desired insecticidal activity. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. For example, the activity can be evaluated by a bioassay in which the lipase and/or Bt insecticidal protein is added to the diet of corn rootworm larvae as described in Example 1. See, for example, Rose and McCabe (1973) J. Econ. Entomol. 66:393, herein incorporated by reference in its entirety.

Variant nucleotide sequences and proteins also encompass sequences and proteins derived from a mutagenic and recombinogenic procedure such as DNA shuffling. With such a procedure, one or more different insecticidal lipase or Bt insecticidal protein encoding sequences can be manipulated to create a new lipase or Bt insecticidal protein possessing the desired insecticidal properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest, such as the catalytic triad and variants thereof, may be shuffled between SEQ ID NO:1 of the invention and other known lipase genes to obtain a new gene coding for a protein with an improved property of interest, such as an increased insecticidal activity. Alternatively, the Bt toxin domain and one of the sequences as set forth in SEQ ID NO:15, SEQ ID NO:17, and/or SEQ ID NO:19, could be shuffled with another toxin protein domain of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

As one example, altered substrate specificity could be one parameter for selection of products of gene shuffling. Lipid acyl hydrolases comprise a diverse multigene family that is conserved across many species. The enzymes exhibit hydrolyzing activity for many glyco- and phospholipids. Substrates include monogalactosyldiacylglycerol, acylsterylgucoside, phosphatidylcholine, lysophosphatidylcholine, phosphatidylethanolamine, lysophosphatidylethanolamine, phosphatidylinositol, as well as many other lipid substrates. Similarly membrane composition of various insects as well as plants can vary from species to species and can be affected by diet or growth conditions. Consequently, the activity of a given lipid acyl hydrolase for a given substrate could affect both specificity and potency.

Solubility and protein stability could also be selected from shuffled gene products. Insecticidal proteins are active in the harsh environment of the insect gut lumen. Their proteins are digested by proteases, and affected by reducing or oxidizing conditions that vary according to the insect species tested. Some forms of Bt insecticidal protein require proteolytic processing in the midgut of the insect before becoming active. Thus, the solubility and stability of either insecticidal lipases or Bt insecticidal proteins in the transgenic plant and in the insect gut lumen could affect biological activity and could be altered through gene shuffling strategies.

Further, conditions for enzyme reactions such as pH and temperature optima may also affect the insecticidal activity of a lipase or Bt insecticidal protein. For example, the gut pH of corn rootworm is 5.5-6.0. Selection of shuffled gene products for enzymatic activity toward lipid substrates or Bt toxin receptors in this pH range is another parameter that could affect toxicity.

Variants of an insecticidal lipase or Bt insecticidal protein should retain the desired biological activity of the native sequence, i.e., pesticidal activity. Methods are available in the art for determining whether a variant polypeptide retains the desired biological activity of the native polypeptide. Biological activity can be measured using bioassays specifically designed for measuring activity of the native polypeptide or protein. See, for example, Czapla and Lang (1990) J. Econ. Entomol. 83(6): 2480-2485; Andrews et al. (1988) Biochem J 252:199-206; and U.S. Pat. No. 5,743,477, all of which are herein incorporated by reference in their entirety. Additionally, antibodies raised against the native sequence polypeptide can be tested for their ability to bind to the variant polypeptide, where effective binding is indicative of a polypeptide having a conformation similar to that of the native polypeptide.

The following terms are used to describe the sequence relationships between two or more polynucleotides or polypeptides: (a) “reference sequence”, (b) “comparison window”, (c) “sequence identity”, and, (d) “percentage of sequence identity.”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program,” it is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The coding sequences for insecticidal lipases and Bt insecticidal proteins encompassed by the invention can be provided in expression cassettes for co-expression in the plant or organism of interest. The cassette may include 5′ and 3′ regulatory sequences operably linked to a polynucleotide encoding an insecticidal lipase, such as an acyl lipid hydrolase, and/or a Bt insecticidal protein. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain additional genes to be cotransformed into the organism. Alternatively, the additional gene(s) can be provided on multiple expression cassettes. Such an expression cassette is provided with a plurality of restriction sites and/or recombination sites for insertion of a polynucleotide that encodes an insecticidal lipase, such as a lipid acyl hydrolase, and or a Bt insecticidal protein, so that the gene is (or genes are) under the transcriptional regulation of the regulatory regions. The expression cassette may additionally contain selectable marker genes.

Such expression cassettes are provided with a plurality of restriction sites for insertion of the lipase and Bt insecticidal protein sequence to be under the transcriptional regulation of the regulatory regions. The expression cassettes may additionally contain selectable marker genes.

The expression cassette will include in the 5′-3′ direction of transcription, a transcriptional and translational initiation region (i.e., a promoter), a DNA sequence encompassed by the invention, such as an insecticidal lipase-encoding DNA sequence and/or a Bt insecticidal protein encoding sequence, and a transcriptional and translational termination region (i.e., termination region) functional in plants. The regulatory regions 10 (i.e., promoters, transcriptional regulatory regions, and translational termination regions) and/or the lipase-encoding polynucleotides and/or Bt insecticidal protein encoding polynucleotides useful in the invention may be native/analogous to the host cell or to each other. Alternatively, the regulatory regions and/or the lipase-encoding polynucleotides and/or Bt insecticidal protein encoding polynucleotides useful in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked lipase-encoding polynucleotides of interest and/or Bt insecticidal protein encoding polynucleotides of interest, may be native with the plant host, or may be derived from another source (i.e., foreign or heterologous to the promoter, the insecticidal lipase-encoding polynucleotide and/or Bt insecticidal protein encoding polynucleotides of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Those skilled in the art recognize that the native DNA sequence encoding the lipase or Bt sequences may not express properly in plants. Therefore, certain modifications to the DNA sequence may be necessary to ensure proper protein expression and folding. For example, Candida cylindracea has unusual codon usage. It translates the codon CTG as a serine instead of the usual leucine as in other organisms, see Kwaguchi et al. (1989), Nature 6238:164-166. As a consequence, if one attempted to express the native DNA sequence in plants, the enzyme would contain leucines instead of serines. In some instances, this substitution might not affect enzymatic activity. However, because the catalytic triad requires a serine in the active site, the serine-to-leucine substitution renders the native-encoded lipase inactive in plants. Thus, replacing the CTG codon with a codon that is read as a serine in plants restores activity. For example, substituting CTG with the codons TCT, TCC, TCA, TCG, AGT, or AGC will cause the plant to translate the correct amino acid—serine—instead of leucine. The DNA sequence set forth in SEQ ID NO:1, which was derived from Candida cylindracea, includes these advantageous substitutions.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids encoding the insecticidal lipase and Bt insecticidal protein can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Generally, it will be beneficial to express the insecticidal protein sequences from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used to drive expression of the insecticidal proteins. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of an insecticidal protein sequence in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced insecticidal lipase and Bt insecticidal protein expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6): 1129-1138; Matsuoka et al. (1993) Proc. Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10): 1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1): 11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference.

Where low level expression is desired, weak promoters will be used to drive expression of the insecticidal protein sequences. Generally, a “weak promoter” is intended to mean a promoter that drives expression of a coding sequence at a low level. By low level expression, levels of about 1/1000 transcripts to about 1/100,000 transcripts to about 1/500,000 transcripts is intended. Alternatively, it is recognized that weak promoters also encompass promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol. Bioeng. 85:610-9 and Fetter et al. (2004) Plant Cell 16:215-28), cyan fluorescent protein (CYP) (Bolte et al. (2004) J. Cell Science 117:943-54 and Kato et al. (2002) Plant Physiol. 129:913-42), and yellow fluorescent protein (PhiYFP™ from Evrogen; see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Nail. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptide gains access to the interior of at least one cell of the plant. Methods for introducing polynucleotides or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotide constructs of the invention may be introduced into plants by contacting plants with a virus or viral nucleic acids. Generally, such methods involve incorporating a nucleotide construct of the invention within a viral DNA or RNA molecule. It is recognized that an insecticidal lipase and/or Bt insecticidal protein useful in the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

In specific embodiments, the insecticidal lipase sequences and Bt insecticidal proteins useful in the invention, can be provided to a plant using a variety of transient transformation methods. Such methods include, for example, microinjection or particle bombardment. See, for example, Crossway et al. (1986) Mol. Gen. Genet. 202:179-185; Nomura et al. (1986) Plant Sci. 44:53-58; Hepler et al. (1994) Proc. Natl. Acad. Sci. 91: 2176-2180 and Hush et al. (1994) J. Cell Sci. 107:775-784, all of which are herein incorporated by reference. Alternatively, an insecticidal lipase-encoding polynucleotide and/or Bt insecticidal protein-encoding polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

Methods are known in the art for the targeted insertion of a polynucleotide at a specific location in the plant genome. In one embodiment, the insertion of the polynucleotide at a desired genomic location is achieved using a site-specific recombination system. See, for example, WO 99/25821, WO 99/25854, WO 99/25840, WO 99/25855, and WO 99/25853, all of which are herein incorporated by reference.

Briefly, the polynucleotide of the invention can be contained in a transfer cassette flanked by two non-recombinogenic recombination sites. The transfer cassette is introduced into a plant having stably incorporated into its genome a target site that is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

In some embodiments, the combination of nucleotide sequences to be introduced into a plant comprises a single nucleotide sequence encoding an insecticidal lipase and a single nucleotide sequence encoding a Bt insecticidal protein, each on its own expression cassette. Where two cassettes are expressed together in the same plant, the cassettes are referred to herein as “stacked” constructs. These expression cassettes may be on different constructs or on the same construct. Where the expression cassettes are both present on the same construct, the construct is further refered to herein as a “molecular stack” construct. In yet other embodiments, the nucleotide sequence encoding an insecticidal lipase and a nucleotide sequence encoding a Bt insecticidal protein are fused and thus expressed as a fusion polynucleotide in a single expression cassette. Such a construct is referred to herein as a “fusion” construct.

In certain other embodiments, this combination of nucleotide sequences can be stacked with any third (or more) polynucleotide sequences of interest in order to create plants with a desired trait. A trait, as used herein, refers to the phenotype derived from a particular expressed nucleotide sequence or groups of sequences. For example, the combination of polynucleotides encoding a Bt insecticidal protein and an insecticidal lipase of the present invention may be stacked with any other polynucleotides encoding polypeptides having pesticidal and/or insecticidal activity, such as lectins (Van Damme et al. (1994) Plant Mol. Biol. 24:825 and the like. The combinations generated can also include multiple copies of any one of the polynucleotides of interest.

For example, in one embodiment, at least one copy of the nucleotide sequence encoding the Bt insecticidal protein is expressed in combination with a plurality of copies of the nucleotide sequence encoding the insecticidal lipase. In another embodiment, at least one nucleotide sequence encoding an insecticidal lipase is expressed in combination with a plurality of nucleotide sequences encoding Bt insecticidal proteins.

The combination of polynucleotides encoding the insecticidal proteins can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference.

The combination of polynucleotides encoding the lipase and Bt insecticidal proteins can also be stacked with other traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; genes coding for resistance to herbicides that act to inhibit action of glutamine synthase, such as phosphinothricin or basta (e.g., bar gene); genes coding for glyphosate resistance (for example, the EPSPS gene and the GAT gene; see, for example, U.S. Publication No. 20040082770 and WO 03/092360)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine the polynucleotides of the present invention with polynucleotides providing agronomic traits such as male sterility (e.g., see U.S. Pat. No. 5,583,210), stalk strength, flowering time, or transformation technology traits such as cell-cycle regulation or gene targeting (e.g., WO 99/61619 WO 00/17364, and WO 99/25821); the disclosures of which are herein incorporated by reference.

These stacked combinations, including the combination of polynucleotides comprising sequences encoding the insecticidal lipase and Bt insecticidal protein, can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 99/25821, WO 99/25854, WO 99/25840, WO 99/25855, and WO 99/25853, all of which are herein incorporated by reference.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Nucleotide sequences encoding insecticidal lipases and Bt insecticidal protein can be manipulated and used to express the proteins in a variety of hosts including, but not limited to, microorganisms and plants. Further, the present invention may be used for transformation of any plant species of interest, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago saliva), rice (Oryza saliva), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The methods of the invention can be utilized to protect plants from pests, especially insect pests. In particular, proteins and nucleotide sequences that are inhibitory or toxic to insects of the order Coleoptera can be obtained and utilized in the methods of the invention. The embodiments of the present invention may be effective against a variety of pests. For purposes of the present invention, pests include, but are not limited to, insects, fungi, bacteria, nematodes, acarids, protozoan pathogens, animal-parasitic liver flukes, and the like.

In particular, proteins and nucleotide sequences which are inhibitory or toxic to insect pests are encompassed by the invention. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of particular relevance include those that infest the major crops. For example: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

It is recognized that having discovered the beneficial effects of coexpression of these two classes of insecticidal proteins, similar protection from insect pests could be accomplished by their co-application to the environment of the target pest(s). Thus, at least one insecticidal lipase and at least one Bt insecticidal protein can be used together to protect plants, seeds, and plant products from insect pests in a variety of ways. When used for co-application, the two classes of insecticidal proteins can be applied as a single pesticidal composition; alternatively, they can be co-applied as two separate pesticidal compositions, one comprising an effective amount of the insecticidal lipase, the other comprising an effective amount of the Bt insecticidal protein. Where more than one member of either class of insecticidal proteins is used to practice the invention, the additional member(s) can be applied as a separate pesticidal composition, or as part of the pesticidal composition comprising either the other insecticidal lipase(s), the other Bt insecticidal protein(s), or the other insecticidal lipase(s) and the other Bt insecticidal protein(s) (i.e., all members of these two classes of insecticidal proteins being co-applied as a single pesticidal composition). For example, the insecticidal lipase and Bt insecticidal protein can be used in a method that involves placing an effective amount of one or more pesticidal compositions that comprise both the lipase and Bt insecticidal proteins in the environment of the pest by a procedure selected from the group consisting of spraying, dusting, broadcasting, or seed coating.

Before plant propagation material (fruit, tuber, bulb, corm, grains, seed), but especially seed, is sold as a commercial product, it is customarily treated with a protectant coating comprising herbicides, insecticides, fungicides, bactericides, nematicides, molluscicides, or mixtures of several of these preparations, if desired together with further carriers, surfactants, or application-promoting adjuvants customarily employed in the art of formulation to provide protection against damage caused by bacterial, fungal, or animal pests. In order to treat the seed, the protectant coating may be applied to the seeds either by impregnating the tubers or grains with a liquid formulation or by coating them with a combined wet or dry formulation. In addition, in special cases, other methods of application to plants are possible, e.g., treatment directed at the buds or the fruit.

The plant seed of the invention comprising a combination of polynucleotides encoding An insecticidal lipase and Bt insecticidal protein may be treated with a seed protectant coating comprising a seed treatment compound, such as, for example, captan, carboxin, thiram, methalaxyl, pirimiphos-methyl, and others that are commonly used in seed treatment. In one embodiment within the scope of the invention, a seed protectant coating comprising a pesticidal composition that comprises both an insecticidal lipase and Bt insecticidal protein is used alone or in combination with one of the seed protectant coatings customarily used in seed treatment.

It is recognized that the polynucleotides comprising sequences encoding the insecticidal lipases and Bt insecticidal proteins can be used to transform insect pathogenic organisms to provide for host organism production of these insecticidal proteins, and subsequent application of the host organism to the environment of the target pest(s). Such host organisms include baculoviruses, fungi, protozoa, bacteria, and nematodes. Optimally, the host organism is co-transformed with polynucleotides comprising the coding sequences for both the insecticidal lipase and Bt insecticidal protein to ensure coexpression of these proteins and maximum exposure to the combination of their pesticidal activities. Alternatively, the individual classes of insecticidal proteins can be expressed in different cohorts of the same host organism, or in different host organisms, with subsequent co-application of the different cohorts or different host organisms to the environment of the target pest(s), so long as expression of these two classes of insecticidal proteins within the different cohorts or different host organisms provides for the combined presentation of both classes of insecticidal proteins to the environment of the target pest(s).

In this manner, the combination of polynucleotides encoding the insecticidal lipase and Bt insecticidal protein may be introduced via a suitable vector into a microbial host, and said host applied to the environment, or to plants or animals. The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

Microorganism hosts that are known to occupy the “phytosphere” (phylloplane, phyllosphere, rhizosphere, and/or rhizoplana) of one or more crops of interest may be selected. These microorganisms are selected so as to be capable of successfully competing in the particular environment with the wild-type microorganisms, provide for stable maintenance and expression of the sequences encoding the lipase and Bt insecticidal proteins, and desirably, provide for improved protection of these insecticidal proteins from environmental degradation and inactivation.

Such microorganisms include bacteria, algae, and fungi. Of particular interest are microorganisms such as bacteria, e.g., Pseudomonas, Erwinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes, fungi, particularly yeast, e.g., Saccharomyces, Cryptococcus, Kluyveromyces, Sporobolomyces, Rhodotorula, and Aureobasidium. Of particular interest are such phytosphere-bacterial species as Pseudomonas syringae, Pseudomonas fluorescens, Serratia marcescens, Acetobacter xylinum, Agrobacteria, Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans. Of particular interest are the pigmented microorganisms.

A number of ways are available for introducing the combination of polynucleotides comprising sequences encoding the lipase and Bt insecticidal proteins into the microorganism host under conditions that allow for stable maintenance and expression of these nucleotide encoding sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (2000); Molecular Cloning: A Laboratory Manual (3^{rd} ed.; Cold Spring Harbor Laboratory Press, Plainview, N.Y.); Davis et al. (1980) Advanced Bacterial Genetics (Cold Spring Harbor Laboratory, Cold Spring Harbor, N.Y.; and the references cited therein.

Suitable host cells, where the pesticidal protein-containing cells will be treated to prolong the activity of the insecticidal proteins in the cell when the treated cell is applied to the environment of the target pest(s), may include either prokaryotes or eukaryotes, normally being limited to those cells that do not produce substances toxic to higher organisms, such as mammals. However, organisms that produce substances toxic to higher organisms could be used, where the toxin is unstable or the level of application sufficiently low as to avoid any possibility of toxicity to a mammalian host. As hosts, of particular interest will be the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

Characteristics of particular interest in selecting a host cell for purposes of pesticidal protein production include ease of introducing the pesticidal protein coding sequence into the host, availability of expression systems, efficiency of expression, stability of the protein in the host, and the presence of auxiliary genetic capabilities. Characteristics of interest for use as a pesticide microcapsule include protective qualities for the pesticide, such as thick cell walls, pigmentation, and intracellular packaging or formation of inclusion bodies; leaf affinity; lack of mammalian toxicity; attractiveness to pests for ingestion; ease of killing and fixing without damage to the toxin; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonas fluorescens, Saccharomyces cerevisiae, Bacillus thuringiensis, Escherichia coli, Bacillus subtilis, and the like.

The combination of polynucleotides comprising sequences encoding the lipase and Bt insecticidal proteins encompassed by the invention can be introduced into microorganisms that multiply on plants (epiphytes) to deliver these two classes of insecticidal proteins to potential target pests. Epiphytes, for example, can be gram-positive or gram-negative bacteria.

Root-colonizing bacteria, for example, can be isolated from the plant of interest by methods known in the art. Specifically, a Bacillus cereus strain that colonizes roots can be isolated from roots of a plant (see, for example, Handelsman et al. (1991) Appl. Environ. Microbiol. 56:713-718). The combination of polynucleotides comprising sequences encoding the lipase and Bt insecticidal proteins can be introduced into a root-colonizing Bacillus cereus by standard methods known in the art.

For example, sequences encoding these insecticidal proteins can be introduced, for example, into the root-colonizing Bacillus by means of electrotransformation. Specifically, nucleotide sequences encoding the lipase and Bt insecticidal proteins can be cloned into a shuttle vector, for example, pHT3101, and the shuttle vector can be transformed into the root-colonizing Bacillus by means of electroporation (Lerecius et al. (1989) FEMS Microbiol. Letts. 60:211-218).

Expression systems can be designed so that these insecticidal proteins are secreted outside the cytoplasm of gram-negative bacteria, E. coli, for example. Advantages of having lipase and Bt insecticidal proteins secreted are: (1) avoidance of potential cytotoxic effects of the pesticidal protein expressed; and (2) improvement in the efficiency of purification of these insecticidal proteins, including, but not limited to, increased efficiency in the recovery and purification of the protein per volume cell broth and decreased time and/or costs of recovery and purification per unit protein.

Insecticidal proteins can be made to be secreted in E. coli, for example, by fusing an appropriate E. coli signal peptide to the amino-terminal end of the insecticidal protein. Signal peptides recognized by E. coli can be found in proteins already known to be secreted in E. coli, for example the OmpA protein (Ghrayeb et al. (1984) EMBO J. 3:2437-2442). OmpA is a major protein of the E. coli outer membrane, and thus its signal peptide is thought to be efficient in the translocation process. Also, the OmpA signal peptide does not need to be modified before processing as may be the case for other signal peptides, for example lipoprotein signal peptide (Duffaud et al. (1987) Meth. Enzymol. 153:492).

The lipase and Bt insecticidal proteins can be fermented in a bacterial host and the resulting bacteria processed and used as a microbial spray in the same manner that Bacillus thuringiensis strains have been used as insecticidal sprays. In the case of an insecticidal protein(s) that is secreted from Bacillus, the secretion signal is removed or mutated using procedures known in the art. Such mutations and/or deletions prevent secretion of the insecticidal protein(s) into the growth medium during the fermentation process. The insecticidal proteins are retained within the cells, and the cells are then processed to yield the encapsulated insecticidal proteins. Any suitable microorganism can be used for this purpose. Pseudomonas has been used to express Bacillus thuringiensis endotoxins as encapsulated proteins and the resulting cells processed and sprayed as an insecticide Gaertner et al. (1993), in Advanced Engineered Pesticides, ed. L. Kim (Marcel Decker, Inc.).

Alternatively, the insecticidal proteins are produced by introducing heterologous genes into a cellular host. Expression of the heterologous genes results, directly or indirectly, in the intracellular production and maintenance of these insecticidal proteins. These cells are then treated under conditions that prolong the activity of the lipase and Bt insecticidal gene produced in the cell when the cell is applied to the environment of target pest(s). The resulting product retains the pesticidal activity of these insecticidal proteins. These naturally encapsulated pesticidal proteins may then be formulated in accordance with conventional techniques for application to the environment hosting a target pest, e.g., soil, water, and foliage of plants. See, for example, EPA 0192319, and the references cited therein.

In the present invention, a transformed microorganism (which includes whole organisms, cells, spore(s), insecticidal protein(s), pesticidal component(s), pest-impacting component(s), mutant(s), optimally living or dead cells and cell components, including mixtures of living and dead cells and cell components, and including broken cells and cell components) or an isolated insecticidal protein can be formulated with an acceptable carrier into separate or combined pesticidal compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present invention (i.e., the combination of at least one lipase and at least one Bt insecticidal protein) are normally applied in the form of compositions and can be applied to the crop area, plant, or seed to be treated. For example, the pesticidal compositions may be applied to grain in preparation for or during storage in a grain bin or silo, etc. The pesticidal compositions may be applied simultaneously or in succession with other compounds. Methods of applying an active ingredient or a pesticidal composition that contains at least one lipase and/or Bt insecticidal protein include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The pesticidal compositions comprising the lipase and/or Bt insecticidal proteins can be in a suitable form for direct application or as a concentrate of primary composition that requires dilution with a suitable quantity of water or other dilutant before application. The pesticidal concentration will vary depending upon the nature of the particular formulation, specifically, whether it is a concentrate or to be used directly. The composition contains 1 to 98% of a solid or liquid inert carrier, and 0 to 50%, optimally 0.1 to 50% of a surfactant. These compositions will be administered at the labeled rate for the commercial product, optimally about 0.01 lb.-5.0 lb. per acre when in dry form and at about 0.01 pts.-10 pts. per acre when in liquid form.

In a further embodiment, the pesticidal compositions, as well as the transformed microorganisms capable of expressing the lipase and Bt insecticidal proteins, can be treated prior to formulation to prolong the pesticidal activity when applied to the environment of a target pest as long as the pretreatment is not deleterious to the activity. Such treatment can be by chemical and/or physical means as long as the treatment does not deleteriously affect the properties of the composition(s). Examples of chemical reagents include, but are not limited to, halogenating agents; aldehydes such a formaldehyde and glutaraldehyde; anti-infectives, such as zephiran chloride; alcohols, such as isopropanol and ethanol; and histological fixatives, such as Bouin's fixative and Helly's fixative (see, for example, Humason (1967) Animal Tissue Techniques (W.H. Freeman and Co.)).

In other embodiments of the invention, it may be advantageous to treat the Bt insecticidal proteins with a protease, for example trypsin, to activate the protein prior to application of a pesticidal protein composition comprising this class of insecticidal proteins to the environment of the target pest. Methods for the activation of protoxin by a serine protease are well known in the art. See, for example, Cooksey (1968) Biochem. J. 6:445-454 and Carroll and Ellar (1989) Biochem. J. 261:99-105, the teachings of which are herein incorporated by reference. For example, a suitable activation protocol includes, but is not limited to, combining a polypeptide to be activated, and trypsin at a 1/100 weight ratio of Bt protein/trypsin in 20 nM NaHCO₃, pH 8 and digesting the sample at 36° C. for 3 hours.

The pesticidal compositions (including the transformed microorganisms) can be applied to the environment of an insect pest by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the pesticidal composition(s) and/or transformed microorganism(s) may be mixed with grain to protect the grain during storage. It is generally important to obtain good control of pests in the early stages of plant growth, as this is the time when the plant can be most severely damaged. The pesticidal compositions can conveniently contain another insecticide if this is thought necessary. In an embodiment of the invention, the pesticidal composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, a herbicide, an insecticide, a fertilizer, in an inert carrier, and dead cells of a Bacillus strain or transformed microorganism of the invention.

The following examples are offered by way of illustration and not by way of limitation.

### EXPERIMENTAL

### Example 1

### Effect of the Combination of an Insecticidal Lipase and Bt Insecticidal Protein on Diabrotica Larvae

Insect diets for southern corn rootworm and western corn rootworm larvae are known in the art. See, for example, Rose and McCabe (1973) J. Econ. Entomology 66:393, herein incorporated by reference. Insect diet was prepared and poured onto a tray. 1.5 ml of diet was dispensed into each cell with an additional 50 μl of sample preparation containing the insecticidal lipase and Bt insecticidal protein of interest applied to the diet surface. Alternatively, 50 μl of PBS buffer adjusted for ammonium sulfate concentration was applied to the control group diet.

For the screening of western corn rootworm, 50 μl of a 0.8 egg agar solution was applied to lids. Trays were allowed to dry under a hood. After drying, lids were placed on trays and stored for 3-5 days at a temperature of 26° C. Trays were then scored counting “live” versus “dead” larvae and tabulating the results. The results were expressed as a percentage of mortality.

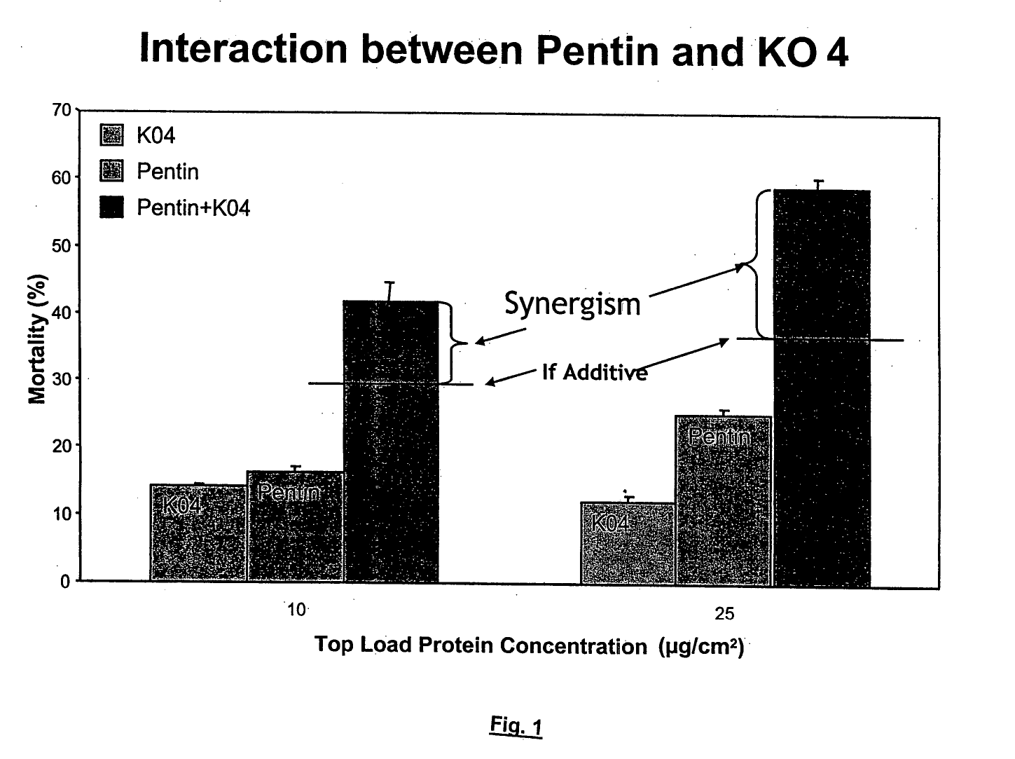

The results of feeding the combination to Diabrotica larvae are shown in FIG. 1 and Table 1. FIG. 1 shows western corn rootworm bioassay results from feeding pentin lipase as set forth in SEQ ID NO:12 and Bt insecticidal protein as set forth in SEQ ID NO:18 to developing larvae. The diet causes a dose-dependent mortality as a percentage of wild-type controls. Table 1 shows the bioassay results from feeding in tabular form. Shown are the mortality scores relative to controls. The combination causes a dose-dependent increase in larval mortality.

TABLE 1

| Pentin | K04 (μg/cm²) | | | | |
|---|---|---|---|---|---|
| (μg/cm²) | 0 | 10 | 25 | 50 | 100 |
| 0 | *8 | 14 | 12 | 22 | 44 |
| 10 | 16 | 42 | 75 | 53 | 69 |
| 25 | 25 | 53 | 59 | 67 | 76 |
| 50 | 35 | 46 | 62 | 58 | 74 |
| 100 | 32 | 52 | 58 | 61 | 73 |

*Values represent average percent mortality over 3 repeats in WCRW bioassay.

### Example 2

### Plasmid Construction

A plasmid vector comprising the sequence set forth in SEQ ID NO:11 operably linked to a ubiquitin promoter (RB-ubi-pentin-PinII) and another operably linked to a rice actin promoter (RB-riceActin-pentin-PinII), and a plasmid vector comprising the sequence set forth in SEQ ID NO:19 operably linked to a ubiquitin promoter (Ubi-1218K054B-PinII: δ 35s-pat-35s-LB) were made.

### Example 3

### Transformation and Regeneration of Transgenic Plants

Immature maize embryos from greenhouse donor plants are bombarded with a plasmid comprising the sequence set forth in SEQ ID NO:11 operably linked to a ubiquitin promoter or rice actin promoter in combination with a plasmid comprising the sequence set forth in SEQ ID NO:19 operably linked to a ubiquitin promoter. A selectable marker gene such as PAT (Wohlleben et al. (1988) Gene 70:25-37), which confers resistance to the herbicide Bialaphos, is used. Alternatively, the selectable marker gene is provided on a separate plasmid. Transformation is performed as follows. Media recipes follow below.

Prior to transformation, the ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

These plasmids plus plasmid DNA containing a PAT selectable marker are precipitated onto 1.1 μm (average diameter) tungsten pellets using a CaCl₂ precipitation procedure as follows:
100 μl prepared tungsten particles in water
10 μl (1 μg) DNA in Tris EDTA buffer (1 μg total DNA)
100 μl 2.5 M CaCl₂
10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun #HE34-1 or #HE34-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for insecticidal activity.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-1H₂O following adjustment to pH 5.8 with KOH); 2.0 gA Gelrite (added after bringing to volume with D-1H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-1H₂O following adjustment to pH 5.8 with KOH); 3.0 μl Gelrite (added after bringing to volume with D-1H₂O); and 0.85 mg/l silver nitrate and 3.0 mg/l bialaphos (both added after sterilizing the medium and cooling to room temperature).

Plant regeneration medium (288J) comprises 4.3 μl MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-1H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-1H₂O), sterilized and cooled to 60° C.

### Example 4

### Agrobacterium-Mediated Transformation

For Agrobacterium-mediated transformation of maize with a combination of an expression cassette comprising SEQ ID NO:11 operably linked to a ubiquitin promoter or a rice actin promoter and an expression cassette comprising SEQ ID NO:19 operably linked to a ubiquitin promoter, the method of Zhao can be employed (U.S. Pat. No. 5,981,840, and International Patent Publication No. WO 98/32326; the contents of which are hereby incorporated by reference). Briefly, immature embryos are isolated from maize and the embryos contacted with a suspension of Agrobacterium, where the bacteria are capable of transferring the combination of a lipase expression cassette and a insecticidal Bt protein expression cassette to at least one cell of claim at least one of the immature embryos (step 1: the infection step). In this step the immature embryos are generally immersed in an Agrobacterium suspension for the initiation of inoculation. The embryos are co-cultured for a time with the Agrobacterium (step 2: the co-cultivation step). Generally, the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Generally, the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Generally, the immature embryos are cultured on solid medium with a selective agent resulting in the selective growth of transformed cells. The callus is then regenerated into plants (step 5: the regeneration step), and, generally, calli grown on selective medium are cultured on solid medium to regenerate the plants. Transformed plants are then grown and selected according to the methods in Example 3.

### Example 5

### Soybean Embryo Transformation

Soybean embryos are bombarded with a combination of plasmids, one having the expression cassette comprising SEQ ID NO:11 operably linked to a ubiquitin promoter or a rice actin promoter and the other having an expression cassette comprising SEQ ID NO:19 operably linked to a ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic PDS 1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising SEQ ID NO:11 operably linked to the ubiquitin promoter and the expression cassette comprising SEQ ID NO:19 operably linked to the ubiquitin promoter the can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1M), and 50 μl CaCl₂ (2.5M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Sunflower Meristem Tissue Transformation

Sunflower meristem tissues are transformed with an expression cassette comprising SEQ ID NO:11 operably linked to the ubiquitin promoter or a rice actin promoter and the expression cassette comprising SEQ ID NO:19 operably linked to the ubiquitin promoter as follows (see also, European Patent Number EP 0 486233, herein incorporated by reference, and Malone-Schoneberg et al. (1994) Plant Science 103:199-207). Mature sunflower seeds (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant. 15:473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney et al. (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the lipase expression cassette and a Bt toxin expression cassette is introduced into Agrobacterium strain EHA105 via freeze-thawing as described by Holsters et al. (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD600 of about 0.4 to 0.8. The Agrobacterium cells are pelleted and resuspended at a final OD600 of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by lipase assay and Bt insecticidal protein bioassay. See, for example, U.S. Pat. No. 5,743,477 herein incorporated by reference in its entirety, and Hosteller et al. (1991) Methods Enzymol. 197:125-134, and Rose and McCabe (1973) J. Econ. Entomology 66:393.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA and/or by lipase and Bt toxin activity analysis of leaf extracts while transgenic seeds harvested from NPTII-positive To plants are identified by lipase activity analysis and Bt toxin analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 μl Bactopeptone, and 5 g/l NaCl, pH 7.0) in the presence of 50 μg/l kanamycin is resuspended in an inoculation medium (12.5 mM 2-mM 2-(N-morpholino)ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour-day and 26° C. incubation conditions.

Explants (around 2 cm long) from two weeks of culture in 374C medium are screened for lipase and Bt toxin activity using assays known in the art and disclosed herein. After positive explants are identified, those shoots that fail to exhibit lipase and Bt toxin activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for lipase and Bt toxin expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for creating or enhancing insect resistance in a plant, said method comprising stably introducing into said plant a combination of polynucleotides, said combination comprising:
a) at least one polynucleotide comprising a sequence encoding a lipase polypeptide having insecticidal activity operably linked to a promoter that drives expression in a plant cell, and
b) at least one polynucleotide comprising a sequence encoding a Bacillus thuringiensis (Bt) insecticidal protein operably linked to a promoter that drives expression in a plant cell.

2. The method of claim 1, wherein at least one polynucleotide of the combination is introduced into said plant through breeding.

3. The method of claim 1, wherein at least one polynucleotide sequence of the combination is introduced into said plant through transformation.

4. The method of claim 1, wherein insect resistance is created or enhanced against any species of the orders selected from the group consisting of Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, and Trichoptera.

5. The method of claim 1, wherein insect resistance is created or enhanced against one or more pests selected from the group consisting of western corn rootworm, northern corn rootworm, southern corn rootworm, Mexican corn rootworm, grubs, and the wireworm.

6. The method of claim 1, wherein said plant is a dicot.

7. The method of claim 1, wherein said plant is a monocot.

8. The method of claim 7, wherein said monocot is maize.

9. The method of claim 1, wherein at least one of said promoters is selected from the group consisting of a constitutive promoter, an inducible promoter, and a tissue-preferred promoter.

10. The method of claim 9, wherein said tissue-preferred promoter is selected from the group consisting of a root-preferred promoter, a leaf-preferred promoter, and a seed-preferred promoter.

11. The method of claim 1, wherein expression of said combination of polynucleotides synergistically enhances insect resistance in said plant.

12. The method of claim 1, wherein said combination of polynucleotides comprises at least one nucleotide sequence selected from the group consisting of:
a) the nucleotide sequence set forth in SEQ ID NO:1, SEQ ID NO:3, SEQ ID NO:5, SEQ ID NO:7, SEQ ID NO:9, SEQ ID NO:1, or SEQ ID NO:13;
b) a nucleotide sequence comprising at least 80% sequence identity to the sequence set forth in SEQ ID NO:1, SEQ ID NO:3, SEQ ID NO:5, SEQ ID NO:7, SEQ ID NO:9, SEQ ID NO:11, or SEQ ID NO:13;
c) a nucleotide sequence comprising at least 15 contiguous nucleotides of the sequence set forth in SEQ ID NO:1, SEQ ID NO:3, SEQ ID NO:5, SEQ ID NO:7, SEQ ID NO:9, SEQ ID NO:11, or SEQ ID NO:13; wherein the sequence encodes a polypeptide having insecticidal activity;
d) a nucleotide sequence encoding a polypeptide comprising the amino acid sequence set forth in SEQ ID NO:2, SEQ ID NO:4, SEQ ID NO:6, SEQ ID NO:8, SEQ ID NO:10, SEQ ID NO:12, or SEQ ID NO:14;
e) a nucleotide sequence encoding a polypeptide having at least 80% sequence identity to the amino acid sequence set forth in SEQ ID NO:2, SEQ ID NO:4, SEQ ID NO:6, SEQ ID NO:8, SEQ ID NO:10, SEQ ID NO:12, or SEQ ID NO:14, wherein said polypeptide has insecticidal activity; and
f) the nucleotide sequence of any one of preceding items (a) through (e), wherein codon usage is optimized for expression in a plant.

13. The method of claim 1, wherein said combination of polynucleotides comprises at least one nucleotide sequence selected from the group consisting of:
a) the nucleotide sequence set forth in SEQ ID NO:15, SEQ ID NO:17, or SEQ ID NO:19;
b) a nucleotide sequence comprising at least 80% sequence identity to the sequence set forth in SEQ ID NO:15, SEQ ID NO:17, or SEQ ID NO:19;
c) a nucleotide sequence comprising at least 15 contiguous nucleotides of the sequence set forth in SEQ ID NO:15, SEQ ID NO:17, or SEQ ID NO:19, wherein the sequence encodes a polypeptide having insecticidal activity;
d) a nucleotide sequence encoding a polypeptide comprising the amino acid sequence set forth in SEQ ID NO:16, SEQ ID NO:18, or SEQ ID NO:20;
e) a nucleotide sequence encoding a polypeptide having at least 80% sequence identity to the amino acid sequence set forth in SEQ ID NO:16, SEQ ID NO:18, or SEQ ID NO:20, wherein said polypeptide has insecticidal activity; and
f) the nucleotide sequence of any one of preceding items (a) through (e), wherein codon usage is optimized for expression in a plant.

14. The method of claim 13, wherein said combination of polynucleotides comprises at least one nucleotide sequence selected from the group consisting of:
a) the nucleotide sequence set forth in SEQ ID NO:1, SEQ ID NO:3, SEQ ID NO:5, SEQ ID NO:7, SEQ ID NO:9, SEQ ID NO:11, or SEQ ID NO:13;
b) a nucleotide sequence comprising at least 80% sequence identity to the sequence set forth in SEQ ID NO:1, SEQ ID NO:3, SEQ ID NO:5, SEQ ID NO:7, SEQ ID NO:9, SEQ ID NO:11, or SEQ ID NO:13;
c) a nucleotide sequence comprising at least 15 contiguous nucleotides of the sequence set forth in SEQ ID NO:1, SEQ ID NO:3, SEQ ID NO:5, SEQ ID NO:7, SEQ ID NO:9, SEQ ID NO:11, or SEQ ID NO:13, wherein the sequence encodes a polypeptide having insecticidal activity;
d) a nucleotide sequence encoding a polypeptide comprising the amino acid sequence set forth in SEQ ID NO:2, SEQ ID NO:4, SEQ ID NO:6, SEQ ID NO:8, SEQ ID NO:10, SEQ ID NO:12, or SEQ ID NO:14;
e) a nucleotide sequence encoding a polypeptide having at least 80% sequence identity to the amino acid sequence set forth in SEQ ID NO:2, SEQ ID NO:4, SEQ ID NO:6, SEQ ID NO:8, SEQ ID NO:10, SEQ ID NO:12, or SEQ ID NO:14, wherein said polypeptide has insecticidal activity; and
f) the nucleotide sequence of any one of preceding items (a) through (e), wherein codon usage is optimized for expression in a plant.

15. A plant comprising a combination of polynucleotides stably integrated into its genome, said combination comprising:
a) at least one polynucleotide comprising a sequence encoding a lipase polypeptide having insecticidal activity operably linked to a promoter that drives expression in a plant cell; and
b) at least one polynucleotide comprising a sequence encoding a Bacillus thuringiensis (Bt) insecticidal protein operably linked to a promoter that drives expression in a plant cell.

16. The plant of claim 15, wherein said combination of polynucleotides comprises at least one nucleotide sequence selected from the group consisting of:
a) the nucleotide sequence set forth in SEQ ID NO:1, SEQ ID NO:3, SEQ ID NO:5, SEQ ID NO:7, SEQ ID NO:9, SEQ ID NO:11, or SEQ ID NO:13;
b) a nucleotide sequence comprising at least 80% sequence identity to the sequence set forth in SEQ ID NO:1, SEQ ID NO:3, SEQ ID NO:5, SEQ ID NO:7, SEQ ID NO:9, SEQ ID NO:11, or SEQ ID NO:13;
c) a nucleotide sequence comprising at least 15 contiguous nucleotides of the sequence set forth in SEQ ID NO:1, SEQ ID NO:3, SEQ ID NO:5, SEQ ID NO:7, SEQ ID NO:9, SEQ ID NO:11, or SEQ ID NO:13; wherein the sequence encodes a polypeptide having insecticidal activity;
d) a nucleotide sequence encoding a polypeptide comprising the amino acid sequence set forth in SEQ ID NO:2, SEQ ID NO:4, SEQ ID NO:6, SEQ ID NO:8, SEQ D NO: 10, SEQ ID NO:12, or SEQ ID NO:14;
e) a nucleotide sequence encoding a polypeptide having at least 80% sequence identity to the amino acid sequence set forth in SEQ ID NO:2, SEQ ID NO:4, SEQ ID NO:6, SEQ ID NO:8, SEQ ID NO:10, SEQ ID NO:12, or SEQ ID NO:14, wherein said polypeptide has insecticidal activity; and
f) the nucleotide sequence of any one of preceding items (a) through (e), wherein codon usage is optimized for expression in a plant.

17. The plant of claim 15, wherein said combination of polynucleotides comprises at least one nucleotide sequence selected from the group consisting of:
a) the nucleotide sequence set forth in SEQ ID NO:15, SEQ ID NO:17, or SEQ ID NO:19;
b) a nucleotide sequence comprising at least 80% sequence identity to the sequence set forth in SEQ ID NO:15, SEQ ID NO:17, or SEQ ID NO:19;
c) a nucleotide sequence comprising at least 15 contiguous nucleotides of the sequence set forth in SEQ ID NO:15, SEQ ID NO:17, or SEQ ID NO:19, wherein the sequence encodes a polypeptide having insecticidal activity;
d) a nucleotide sequence encoding a polypeptide comprising the amino acid sequence set forth in SEQ ID NO:16, SEQ ID NO:18, or SEQ ID NO:20;
e) a nucleotide sequence encoding a polypeptide having at least 80% sequence identity to the amino acid sequence set forth in SEQ ID NO:16, SEQ ID NO:18, or SEQ ID NO:20, wherein said polypeptide has insecticidal activity; and
f) the nucleotide sequence of any one of preceding items (a) through (e), wherein codon usage is optimized for expression in a plant.

18. The plant of claim 17, wherein said combination of polynucleotides comprises at least one nucleotide sequence selected from the group consisting of:
a) the nucleotide sequence set forth in SEQ ID NO:1, SEQ ID NO:3, SEQ ID NO:5, SEQ ID NO:7, SEQ ID NO:9, SEQ ID NO:1, or SEQ ID NO:13;
b) a nucleotide sequence comprising at least 80% sequence identity to the sequence set forth in SEQ ID NO:1, SEQ ID NO:3, SEQ ID NO:5, SEQ ID NO:7, SEQ ID NO:9, SEQ ID NO:1, or SEQ ID NO:13;
c) a nucleotide sequence comprising at least 15 contiguous nucleotides of the sequence set forth in SEQ ID NO:1, SEQ ID NO:3, SEQ ID NO:5, SEQ ID NO:7, SEQ ID NO:9, SEQ ID NO:1, or SEQ ID NO:13; wherein the sequence encodes a polypeptide having insecticidal activity;
d) a nucleotide sequence encoding a polypeptide comprising the amino acid sequence set forth in SEQ ID NO:2, SEQ ID NO:4, SEQ ID NO:6, SEQ ID NO:8, SEQ ID NO:10, SEQ ID NO:12, or SEQ ID NO:14;
e) a nucleotide sequence encoding a polypeptide having at least 80% sequence identity to the amino acid sequence set forth in SEQ ID NO:2, SEQ ID NO:4, SEQ ID NO:6, SEQ ID NO:8, SEQ ID NO:10, SEQ ID NO:12, or SEQ ID NO:14, wherein said polypeptide has insecticidal activity; and
f) the nucleotide sequence of any one of preceding items (a) through (e), wherein codon usage is optimized for expression in a plant.

19. The plant of claim 15, wherein said plant is a monocot.

20. Transformed seed of the plant of claim 15.
